SEQUENCE LISTING (38 sequences)

SEQ ID NO: 1 - DNA, 250 bp, Variovorax paradoxus
cacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaattcgccagagatggcttagtgctcgaaagagaaccgtaacacaggtgctgc

SEQ ID NO: 2 - DNA, 250 bp, Variovorax ginsengisoli
cacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaatttgccagagatggcttagtgctcgaaagagaaccgtaacacaggtgctgc

SEQ ID NO: 3 - DNA, 247 bp, Niastella gongjuensis
cacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgc

SEQ ID NO: 4 - DNA, 249 bp, Streptomyces rishiriensis
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacaccggaaacggccagagatggtcgcccccttgtggtcggtgtacaggtggtgc

SEQ ID NO: 5 - DNA, 247 bp, Ferruginibacter lapsinanis
cacgccctaaactatggatactcgacatcagcgatacactgttggtgtctgagcgaaagcattaagtatcccacctgggaagtacgatcgcaagattgaaactcaaaggaattggcgggggtccgcacaagcggtggagcatgtggtttaattcgatgatacgcgaggaaccttacctgggctagaatgctgggagaccgtgggtgaaagctcactttgtagcaatacactgccagtaaggtgctgc

SEQ ID NO: 6 - DNA, 255 bp, Artificial Sequence
  Synthetic polynucleotide
cacgctgtaaacgatgacgactaggtgtgggggggttcagtcccttccgtgccgcagctaacgcattaagtcgtccgcctggggagtacggccgcaaggttgaaactcaaaggaattgacgggggcccgcacaagcggtggagcatgtggttcaattcgatgcagagcgaagaaccttacctgggcttgacatgtaggaagtagaaaccggaaacggggacgacctgttaagtcaggagcctacacaggtggtgc

SEQ ID NO: 7 - DNA, 249 bp, Streptomyces ossamyceticus
cacgccgtaaacggtgggaactaggtgttggcgacattccacgtcgtcggtgccgcagctaacgcattaagttccccgcctggggagtacggccgcaaggctaaaactcaaaggaattgacgggggcccgcacaagcagcggagcatgtggcttaattcgacgcaacgcgaagaaccttaccaaggcttgacatacgccggaaaaccctggagacagggtcccccttgtggtcggtgtacaggtggtgc

SEQ ID NO: 8 - DNA, 1457 bp, Variovorax paradoxus
attgaacgctggcggcatgccttacacatgcaagtcgaacggcagcgcgggagcaatcctggcggcgagtggcgaacgggtgagtaatacatcggaacgtgcccaatcgtgggggataacgcagcgaaagctgtgctaataccgcatacgatctacggatgaaagcaggggatcgcaagaccttgcgcgaatggagcggccgatggcagattaggtagttggtgaggtaaaggctcaccaagccttcgatctgtagctggtctgagaggacgaccagccacactgggactgagacacggcccagactcctacgggaggcagcagtggggaattttggacaatgggcgaaagcctgatccagccatgccgcgtgcaggatgaaggccttcgggttgtaaactgcttttgtacggaacgaaacggccttttctaataaagagggctaatgacggtaccgtaagaataagcaccggctaactacgtgccagcagccgcggtaatacgtagggtgcaagcgttaatcggaattactgggcgtaaagcgtgcgcaggcggtaatgtaagacagttgtgaaatccccgggctcaacctgggaactgcatctgtgactgcattgctggagtacggtagagggggatggaattccgcgtgtagcagtgaaatgcgtagatatgcggaggaacaccgatggcgaaggcaatcccctggacctgtactgacgctcatgcacgaaagcgtggggagcaaacaggattagataccctggtagtccacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaattcgccagagatggcttagtgctcgaaagagaaccgtaacacaggtgctgcatggctgtcgtcagctcgtgtcgtgagatgttgggttaagtcccgcaacgagcgcaacccttgtcattagttgctacatttagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtcttgtacacaccgcccgtcacaccatgggagcgggttctgccagaagtagttagcctaaccgcaaggagggcgattaccacggcagggttcgtgactggggtg

SEQ ID NO: 9 - DNA, 1356 bp, Variovorax paradoxus
attgaacgctggcggcatgccttacacatgcaagtcgaacggcagcgcgggagcaatcctggcggcgagtggcgaacgggtgagtaatacatcggaacgtgcccaatcgtgggggataacgcagcgaaagctgtgctaataccgcatacgatctacggatgaaagcaggggatcgcaagaccttgcgcgaatggagcggccgatggcagattaggtagttggtgaggtaaaggctcaccaagccttcgatctgtagctggtctgagaggacgaccagccacactgggactgagacacggcccagactcctacgggaggcagcagtggggaattttggacaatgggcgaaagcctgatccagccatgccgcgtgcaggatgaaggccttcgggttgtaaactgcttttgtacggaacgaaacggccttttctaataaagagggctaatgacggtaccgtaagaataagcaccggctaactacgtgccagcagccgcggtaatacgtagggtgcaagcgttaatcggaattactgggcgtaaagcgtgcgcaggcggtaatgtaagacagttgtgaaatccccgggctcaacctgggaactgcatctgtgactgcattgctggagtacggtagagggggatggaattccgcgtgtagcagtgaaatgcgtagatatgcggaggaacaccgatggcgaaggcaatcccctggacctgtactgacgctcatgcacgaaagcgtggggagcaaacaggattagataccctggtagtccacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaattcgccagagatggcttagtgctcgaaagagaaccgtaacacaggtgctgcatggctgtcgtcagctcgtgtcgtgagatgttgggttaagtcccgcaacgagcgcaacccttgtcattagttgctacatttagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtct

SEQ ID NO: 10 - DNA, 1457 bp, Variovorax paradoxus
attgaacgctggcggcatgccttacacatgcaagtcgaacggcagcgcgggagcaatcctggcggcgagtggcgaacgggtgagtaatacatcggaacgtgcccaatcgtgggggataacgcagcgaaagctgtgctaataccgcatacgatctacggatgaaagcaggggatcgcaagaccttgcgcgaatggagcggccgatggcagattaggtagttggtgaggtaaaggctcaccaagccttcgatctgtagctggtctgagaggacgaccagccacactgggactgagacacggcccagactcctacgggaggcagcagtggggaattttggacaatgggcgaaagcctgatccagccatgccgcgtgcaggatgaaggccttcgggttgtaaactgcttttgtacggaacgaaacggccttttctaataaagagggctaatgacggtaccgtaagaataagcaccggctaactacgtgccagcagccgcggtaatacgtagggtgcaagcgttaatcggaattactgggcgtaaagcgtgcgcaggcggtaatgtaagacagttgtgaaatccccgggctcaacctgggaactgcatctgtgactgcattgctggagtacggcagagggggatggaattccgcgtgtagcagtgaaatgcgtagatatgcggaggaacaccgatggcgaaggcaatcccctgggcctgtactgacgctcatgcacgaaagcgtggggagcaaacaggattagataccctggtagtccacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaattcgccagagatggcttagtgctcgaaagagaaccgtaacacaggtgctgcatggctgtcgtcagctcgtgtcgtgagatgttgggttaagtcccgcaacgagcgcaacccttgtcattagttgctacattcagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtcttgtacacaccgcccgtcacaccatgggagcgggttctgccagaagtagttagcttaaccgcaaggagggcgattaccacggcagggttcgtgactggggtg

SEQ ID NO: 11 - DNA, 1356 bp, Variovorax paradoxus
attgaacgctggcggcatgccttacacatgcaagtcgaacggcagcgcgggagcaatcctggcggcgagtggcgaacgggtgagtaatacatcggaacgtgcccaatcgtgggggataacgcagcgaaagctgtgctaataccgcatacgatctacggatgaaagcaggggatcgcaagaccttgcgcgaatggagcggccgatggcagattaggtagttggtgaggtaaaggctcaccaagccttcgatctgtagctggtctgagaggacgaccagccacactgggactgagacacggcccagactcctacgggaggcagcagtggggaattttggacaatgggcgaaagcctgatccagccatgccgcgtgcaggatgaaggccttcgggttgtaaactgcttttgtacggaacgaaacggccttttctaataaagagggctaatgacggtaccgtaagaataagcaccggctaactacgtgccagcagccgcggtaatacgtagggtgcaagcgttaatcggaattactgggcgtaaagcgtgcgcaggcggtaatgtaagacagttgtgaaatccccgggctcaacctgggaactgcatctgtgactgcattgctggagtacggcagagggggatggaattccgcgtgtagcagtgaaatgcgtagatatgcggaggaacaccgatggcgaaggcaatcccctgggcctgtactgacgctcatgcacgaaagcgtggggagcaaacaggattagataccctggtagtccacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaattcgccagagatggcttagtgctcgaaagagaaccgtaacacaggtgctgcatggctgtcgtcagctcgtgtcgtgagatgttgggttaagtcccgcaacgagcgcaacccttgtcattagttgctacattcagttgggcactctaatgagactgccggtgacaaaccggaggaaggtggggatgacgtcaagtcctcatggcccttataggtggggctacacacgtcatacaatggctggtacaaagggttgccaacccgcgagggggagctaatcccataaaaccagtcgtagtccggatcgcagtctgcaactcgactgcgtgaagtcggaatcgctagtaatcgtggatcagaatgtcacggtgaatacgttcccgggtct

SEQ ID NO: 12 - DNA, 1451 bp, Niastella gongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcactggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttggggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggtcaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaatgatacggtgaatacgttcccggaccttgcacacaccgcccgtcaagccatggaagctgggtgtacctaaagtcggtaaccgcaaggagccgcctagggtaaaactagtgactggggct

SEQ ID NO: 13 - DNA, 1451 bp, Niastella gongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcaccggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttggggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggtcaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaatgatacggtgaatacgttcccggaccttgcacacaccgcccgtcaagccatggaagctgggtgtacctaaagtcggtaaccgcaaggagccgcctagggtaaaactagtgactggggct

SEQ ID NO: 14 - DNA, 1451 bp, Niastella gongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcaccggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttgaggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggttaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaacgatacggtgaatacgttcccggaccttgcacacaccgcccgtcaagccatggaagctgggtgtacctaaagtcggtaaccgcaaggagccgcctagggtaaaactagtgactggggct

SEQ ID NO: 15 - DNA, 1359 bp, Niastella gongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcactggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttggggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggtcaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaatgatacggtgaatacgttcccggacct

SEQ ID NO: 16 - DNA, 1359 bp, Niastella gongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcaccggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttggggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggtcaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaatgatacggtgaatacgttcccggacct

SEQ ID NO: 17 - DNA, 1359 bp, Niastella gongjuensis
gatgaacgctagcggcaggcttaatacatgcaagtcgaggggcagcgcagtgtagcaatacatgggcggcgaccggcaaacgggtgcggaacacgtacgcaaccttccttcaaatgggggatagcccaccgaaaggtggattaataccccgtaacattttctggtggcatcaccggattattatagctccggcgtttgaagatgggcgtgcgcctgattaggtagttggcggagtaacagcccaccaagcctacgatcagtaactggtgtgagagcacgaccagtcacacgggcactgagacacgggcccgactcctacgggaggcagcagtaaggaatattggtcaatggacgcaagtctgaaccagccatgccgcgtggaggatgaaggtcctctggattgtaaacttcttttatcggagaagaaaccctcaaattcttttgaggttgacggtacccgatgaataagcaccggctaactccgtgccagcagccgcggtaatacggagggtgcaagcgttatccggattcactgggtttaaagggagcgtaggtgggatggtaagtcagtggtgaaatctccgggcttaactcggaaactgccgttgatactatcattcttgaatattgtggaggtaagcggaatatgtcatgtagcggtgaaatgcttagatatgacatagaacaccgatagcgaaggcagcttgctacacaattattgacactgaggctcgaaagcgtggggatcaaacaggattagataccctggtagtccacgccctaaacgatggatactcgacatacgcgatacacagtgtgtgtctgagcgaaagcattaagtatcccacctgggaagtacgaccgcaaggttgaaactcaaaggaattggcgggggtccgcacaagcggtggagcatgtggtttaattcgatgatacgcgaggaaccttacctgggctagaatgcagattgaccgtgggtgaaagctcattttgtagcaatacacagtctgtaaggtgctgcatggctgtcgtcagctcgtgccgtgaggtgttgggttaagtcccgcaacgagcgcaacccccatcattagttgccatcaggttaagctgggaactctaatgaaactgccgtcgtaagacgcgaggaaggaggggatgatgtcaagtcatcatggcctttatgcccagggctacacacgtgctacaatggaagagacaaagagctgccacttggtgacaaggcgctaatctcaaaaactctttctcagttcagatcgcagtctgcaactcgactgcgtgaagctggaatcgctagtaatcgtatatcagcaacgatacggtgaatacgttcccggacct

SEQ ID NO: 18 - DNA, 1448 bp, Arthrobacter globiformis
gatgaacgctggcggcgtgcttaacacatgcaagtcgaacgatgatccggtgcttgcaccggggattagtggcgaacgggtgagtaacacgtgagtaacctgcccttgactctgggataagcctgggaaactgggtctaataccggatatgactcctcatcgcatggtggggggtggaaagcttttgtggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgccgtgaaagtccggggctcaactccggatctgcggtgggtacgggcaggctagagtgatgtaggggagactggaattcctggtgtagcggtgaaatgcgcagatatcaggaggaacaccgatggcgaaggcaggtctctgggcattaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagagatgtggtttctccttttggggccggttcacaggtggtgcatggttgtcgtcagctcgtgtcgtgagatgttgggttaagtcccgcaacgagcgcaaccctcgttccatgttgccagcgcgtaatggcggggactcatgggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggccttgtacacaccgcccgtcaagtcacgaaagttggtaacacccgaagccggtggcctaaccccttgtgggagggagccgtcgaaggtgggactggcgattgggact

SEQ ID NO: 19 - DNA, 1344 bp, Arthrobacter globiformis
gatgaacgctggcggcgtgcttaacacatgcaagtcgaacgatgatccggtgcttgcaccggggattagtggcgaacgggtgagtaacacgtgagtaacctgcccttgactctgggataagcctgggaaactgggtctaataccggatatgactcctcatcgcatggtggggggtggaaagcttttgtggttttggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgcaagcctgatgcagcgacgccgcgtgagggatgacggccttcgggttgtaaacctctttcagtagggaagaagcgaaagtgacggtacctgcagaagaagcgccggctaactacgtgccagcagccgcggtaatacgtagggcgcaagcgttatccggaattattgggcgtaaagagctcgtaggcggtttgtcgcgtctgccgtgaaagtccggggctcaactccggatctgcggtgggtacgggcaggctagagtgatgtaggggagactggaattcctggtgtagcggtgaaatgcgcagatatcaggaggaacaccgatggcgaaggcaggtctctgggcattaactgacgctgaggagcgaaagcatggggagcgaacaggattagataccctggtagtccatgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagagatgtggtttctccttttggggccggttcacaggtggtgcatggttgtcgtcagctcgtgtcgtgagatgttgggttaagtcccgcaacgagcgcaaccctcgttccatgttgccagcgcgtaatggcggggactcatgggagactgccggggtcaactcggaggaaggtggggacgacgtcaaatcatcatgccccttatgtcttgggcttcacgcatgctacaatggccggtacaaagggttgcgatactgtgaggtggagctaatcccaaaaagccggtctcagttcggattggggtctgcaactcgaccccatgaagtcggagtcgctagtaatcgcagatcagcaacgctgcggtgaatacgttcccgggcct

SEQ ID NO: 20 - DNA, 252 bp, Arthrobacter globiformis
catgccgtaaacgttgggcactaggtgtgggggacattccacgttttccgcgccgtagctaacgcattaagtgccccgcctggggagtacggccgcaaggctaaaactcaaaggaattgacgggggcccgcacaagcggcggagcatgcggattaattcgatgcaacgcgaagaaccttaccaaggcttgacatggaccggaccgccgcagagatgtggtttctccttttggggccggttcacaggtggtgc

SEQ ID NO: 21 - DNA, 21 bp, Artificial Sequence
  Primer
ggattagawacccbggtagtc

SEQ ID NO: 22 - DNA, 18 bp, Artificial Sequence
  Primer
tcacrryacgarctgrcg

SEQ ID NO: 23 - DNA, 10 bp, Artificial Sequence
  Primer
agagtttgat

SEQ ID NO: 24 - DNA, 8 bp, Artificial Sequence
  Primer
ggattaga

SEQ ID NO: 25 - DNA, 17 bp, Artificial Sequence
  Primer
  misc_feature (3)..(3): n is a, c, g, or t
  misc_feature (6)..(6): n is a, c, g, or t
  misc_feature (10)..(10): n is a, c, g, or t
tgnacncacngcccgtc

SEQ ID NO: 26 - DNA, 16 bp, Artificial Sequence
  Primer
atggctgtcgtcagct

SEQ ID NO: 27 - DNA, 8 bp, Artificial Sequence
  Primer
tggctcag

SEQ ID NO: 28 - DNA, 7 bp, Artificial Sequence
  Primer
ggtagtc

SEQ ID NO: 29 - DNA, 228 bp, Variovorax sp.
  Marker nucleic acid sequence
tagtatatttttttattttttaaatattaaaaattgagaaattcataaaaaatagaattgacatcaaaaaatcaccattttttgtgtagggccgttaaaagatatgtcaaaattattggagttatcattggtacattttctttaaaagtgcacgctcaacttgtaagatattggcttaaatggctactttgcatgtggtgatcgggctacaatagacaaccaagtagc

SEQ ID NO: 30 - DNA, 296 bp, Variovorax sp.
  Marker nucleic acid sequence
agcacgcggctgcgccacatcatctgcggcggcgaggcgatgccggccgagacgcagaaggagacgctgcaacgcctgcgcggtgcgacgctgcagaacctctacggccccaccgaaaccaccatccacgtcacgcgctggacctgccgtgacaatgggcagagcctcgtgcccatcggccagccgatcaccgacacgcaggcgtacgtgctcgatgccgaactcaacctcgtgccgcgcggcgtggcgggcgagctctacctcggcggcgtgagcctggcgcggggctacctgaa

SEQ ID NO: 31 - DNA, 263 bp, Variovorax sp.
  Marker nucleic acid sequence
catgaattgaaatccttatctatattctcatagggcataaacactgaaaaatatggtcaatattgtgattataatattttttttatagaaaaatgggttttcttaattataattaataaagaaatctacagtcctataacaagacttgcgcaaagcaagaagtctttgcagtgtgacaaggaccttattgcattacagggattgtattgagaaaaaaggaattgggattttctttactccctgtttttgttgtcaaatcaatc

SEQ ID NO: 32 - DNA, 244 bp, Variovorax sp.
  Marker nucleic acid sequence
aattcctaaaagttttatgtacttattcagtaaaattaataagacaattgcttaagtccgatatgccaagaattcttgctaccaaagagataagatcgatggagttaggggctgctttttttttttcttttcttttaaggaaaagattatataaatgaagcaggtacaattagcaataattaataagttcactgctacaattaataaaaaaagaacattatcaacttttgatagaaggattatt

SEQ ID NO: 33 - DNA, 242 bp, Variovorax sp.
  Marker nucleic acid sequence
atcagcagcttgggtccgttgcccgtgcccttgcgcaggccgaccacgttgccttcggcgtcgatcttcgcgtcggcgaggcccagtgccttcatgcgcgcgaggaaggcttcggcgcgcttctgctccttgaacggcggtgcctcgatctcggtcagcatcttgaggtcttcgaccgcgcggtcatggtcggccttgaccgcgtcgagcagtttttggaccgtgggcgccgccatcagctg

SEQ ID NO: 34 - DNA, 490 bp, Variovorax sp.
  Marker nucleic acid sequence
aaagccagcccgaatccggagcggcaggacacattcagggagtcccatgttcgaactacacggcgtgcctgcacgcctgcggcagccatgaagcgccgcgtcatcatccgtacgccgctgggcgaggcactgcaattccaccgcctggccgggcgagagacgctcagccgaccatatgtcttcgatgtcgacctgctgggccgcagcaacgccatcgactccgccgccctgctcggcaagcccgccaccgtggtgatggaaaccgagagcggcgccctgcgctacctctcgggcctggtcacccgcttcggccttgcgcagcaagacgaccgccaggccttctacaagatgcgcctgcgcccgtggctctggctcgccacgcgccgaagcgatttcagggtgtttcaggaccagtccgtgcccgagatcgtcgcggccgtgctgggccgctacggctaccccctcgaacagaagctcagccgcagctacc

SEQ ID NO: 35 - DNA, 472 bp, Variovorax sp.
  Marker nucleic acid sequence
ccgtcgccggcaacctgtccggtgccgatgccggcaactacacgctggcgagcaacgccacgacgcaggccgacatcacggccaaggccatcaccggcagcatcacggccggcaacaaggtctacgacggcaccaccggcgtcgtcaccagtggtgtgctcaacggcctcatcgttggcgacagcgtgagcttcgcgaccagtggcagcttcgctgacaagaatgctggcatcggcaagacggtcaccgtcgccggcagcctgtcgggtgccgatgccggcaactacacgctggggagcaacgatcgcaccacggccgacgtcacgcgccgggcgatcaatggcagcgttaccgcggacaacaagatctacgacggcactaccggcgccaccaccagcggcatgctctacggcctgatcgatggagacagcgtgagcttcgcaaccaccggcagcttcgctgacaagaacgc

SEQ ID NO: 36 - DNA, 352 bp, Variovorax sp.
  Marker nucleic acid sequence
gcgccagtcgctggatgccgcgctgcacccgctgcggctcaatgcggtgttcgaggtcgagcacctggccacggtgatggggctggtcgaggccggcatcggcatcagcgtggtgccggaactcacgctcttccacttccagcgcgagacgctggtgacgcggccgctgccgctgcccggcctcacgcgcaccatctacctggtgcagcgccgggaaggcagcctctcggtggcggcgcagacgctgcacgacctgatcctcgagcggctgggtacgccggcatgaaagttcgtttggaaaaaaagaacccgccgaggcgggttgagaggtagccaaggtgcccattgcagg

SEQ ID NO: 37 - DNA, 283 bp, Variovorax sp.
  Marker nucleic acid sequence
gcggcatcggttcgctgaagcaggatgcaacggtggcgtatgcgttctggcagaaggctgcggacatggggagcatgaatgcacaagcctatttgggcgagaagctgaatgctggctacgatgatcctccttctttctggggcaactatgttgttggccggaagatgctcgaatgtgctttttctcaagggagtggagcagctgcgctagggaaagtctgaacaagtgcacgtttcttgcatgaaggattgacccctgcaaaggaacacgagcgatgagccag

SEQ ID NO: 38 - DNA, 276 bp, Variovorax sp.
  Marker nucleic acid sequence
cgggcgccagtgggtgctgtcgctcacacccgagggcctgcgccggctcaaggcgctgatgcgcgcgcgcgaggccgccttggcccgcgccatcggcgccacgctgagcgccgaacagcgcgcgctgctgctgcaggcctgcgggctgctcgaccgtattgccggcgcgctcgaatgacgtcgatggcaccggccctgcagcgcatcggcggcttctggcgcgagggccgctggcgcgcgggcgcgcagctggcggccgcggtggcgctggcatgg

(19) United States
(12) Patent Application Publication
(10) Pub. No.: US 2022/0132860 A1
(43) Pub. Date: May 5, 2022

(54) PLANT GROWTH-PROMOTING MICROBES, COMPOSITIONS, AND USES THEREOF

(71) Applicant: TAXON BIOSCIENCES INC., St. Louis, MO (US)

(72) Inventors: Michal Shoresh, St. Louis, MO (US); Christine Clarke, St. Louis, MO (US); Crystal Lynn Emery, St. Louis, MO (US); Caroline Kostecki, St. Louis, MO (US); Victor Kunin, St. Louis, MO (US); Honor Renee Lafitte, St. Louis, MO (US); Ulrika Lidstrom, St. Louis, MO (US); Gregory Linshiz, St. Louis, MO (US); Jessica Schaffer, St. Louis, MO (US); Natalia Shestakova, St. Louis, MO (US); Lawrence Kent Wood, St. Louis, MO (US)

(21) Appl. No.: 17/431,708

(22) PCT Filed: Feb. 18, 2020
(86) PCT No.: PCT/IL2020/050180

§ 371 Date: Aug. 17, 2021

Related U.S. Application Data
(60) Provisional application No. 62/807,839, filed on Feb. 20, 2019.

(51) Int. Cl. A01N 63/20; C12N 1/20

(52) U.S. Cl. CPC A01N 63/20; C12R 2001/01; C12N 1/205

(57) ABSTRACT

The present invention relates to isolated plant growth promoting microbes (PGPMs), isolated cultures of these PGPMs and compositions comprising same, including compositions comprising plants or parts thereof, particularly seeds. The present invention further relates to methods of using these isolated PGPMs, isolated cultures and compositions comprising same for enhancing plant health, plant growth and/or plant yield.

### FIELD OF THE INVENTION

The present invention relates to isolated plant growth promoting microbes (PGPMs), isolated cultures thereof and compositions comprising same and use of these PGPMs or composition, for enhancing plant health, plant growth and/or plant yield, and/or for preventing, inhibiting, or treating the development of plant pathogens or the development of phytopathogenic diseases.

### BACKGROUND OF THE INVENTION

Plant growth promoting microbes (PGPMs), such as plant growth-promoting rhizobacteria (PGPR), have gained worldwide importance and acceptance for agricultural benefits. PGPMs can affect plant growth by different direct and indirect mechanisms. There is a considerable amount of ongoing scientific research directed to understanding PGPMs, including the aspects of their adaptation, effects on plant physiology and growth, induced systemic resistance, biocontrol of plant pathogens, bio-fertilization, viability of co-inoculation, interactions with plant microorganisms, and mechanisms of root colonization. For example, International (PCT) Application Publication Nos. WO 2016/044085, WO 2018/208,722 and WO 2019/145,949 to the Applicant of the present invention and others have disclosed various types of PGPMs.

By virtue of their rapid rhizosphere colonization and stimulation of plant growth and/or yield, there is currently considerable interest in exploiting PGPMs to improve crop production and grain yield. In fact, the inoculation of cultivated plants with PGPMs is currently considered a promising agricultural approach. As environmental concerns increase, e.g., concerns about groundwater quality with excess fertilizer and pesticide exposure in foods, biological alternatives are promising and becoming necessary. Thus, developing biological treatments compatible with fertilizers and pesticides and/or even reducing the amount of these chemical compounds used could be a significant advancement in the agricultural industry.

There is a continuing and pressing need for the identification of new PGPMs, PGPM synthetic consortia, and/or testing of their compatibility with existing commercially available crop management products.

### SUMMARY OF THE INVENTION

The present invention addresses the aforementioned need by providing new plant growth promoting microbes (PGPMs), isolates, cultures, compositions, synthetic consortia, and methods useful for enhancing the health, growth and/or yield of a plant. Also provided are methods for the treatment of plants or plant seeds by using the plant growth promoting microbial strains (PGPMs), isolates, cultures or compositions disclosed herein.

This application also provides non-naturally occurring plant varieties that are artificially infected with at least one microbial strain disclosed herein. Other embodiments provide seed, reproductive tissue, vegetative tissue, regenerative tissues, plant parts, or progeny of the non-naturally occurring plant varieties. Other embodiments further provide a method for preparing agricultural compositions.

According to certain aspects, the present invention provides isolated plant growth promoting microbial strains (PGPMs), isolated cultures thereof, biologically pure cultures thereof, and enriched cultures thereof.

According to one aspect, the present invention provides an isolated microbial strain or a functional homolog thereof, wherein the isolated microbial strain is selected from the group consisting of:
(1) strain S3167, the strain being selected from the group consisting of:
   a. a strain deposited under Accession Number NRRL No. B-67735;
   b. a strain comprising at least one 16S-rRNA sequence comprising a nucleic acid sequence selected from the group consisting of SEQ ID NOs:1, 8, and 9; and
   c. a strain comprising at least one genomic marker comprising a nucleic acid sequence selected from the group consisting of SEQ ID NOs:29, 30, 31, 32, and 33;
(2) strain S2492, the strain being selected from the group consisting of:
   a. a strain deposited under Accession Number NRRL No. B-67736;
   b. a strain comprising at least one 16S-rRNA sequence comprising a nucleic acid sequence selected from the group consisting of SEQ ID NOs:1, 10, and 11; and
   c. a strain comprising at least one genomic marker comprising a nucleic acid sequence selected from the group consisting of SEQ ID NOs:34, 35, 36, 37, and 38;
(3) strain S2441, the strain comprising a 16S-rRNA sequence comprising the nucleic acid sequence set forth in SEQ ID NO:2;
(4) strain S2876, comprising a 16S-rRNA sequence comprising the nucleic acid sequence set forth in SEQ ID NO:3;
(5) strain S2550, the strain comprising a 16S-rRNA sequence comprising the nucleic acid sequence set forth in SEQ ID NO:4; and
(6) a strain comprising a 16S-rRNA sequences comprising a nucleic acid sequence selected from the group consisting of SEQ ID NOs:5, 6, or 7.

Each possibility represents a separate embodiment of the present invention.

According to certain embodiments, each of the strains S3167, S2492, and S2441 is a bacterial strain of the genus Variovorax.

According to certain embodiments, each of the strains S3167 and S2492 is of the bacterial species V. paradoxus. According to certain embodiments, the strain S2441 is of the species V. ginsengisoli.

According to certain embodiments, the strain S2876 is a bacterial strain of the species Niastella gongjuensis.

According to certain embodiments, the strain S2550 is a bacterial strain of the species Streptomyces rishiriensis.

According to certain embodiments, the strain comprising a 16S-rRNA sequence having the nucleic acid sequence set forth in SEQ ID NO:5 is a bacterial strain of the species Ferruginibacter lapsinanis.

According to certain embodiments, the strain comprising a 16S-rRNA sequence having the nucleic acid sequence set forth in SEQ ID NO:7 is a bacterial strain of the species Streptomyces ossamycetichs.

According to certain embodiments, the functional homolog of microbial strain S3167 comprises at least one of: a 16S-rRNA sequence at least 85% identical to SEQ ID NO:1, a 16S-rRNA sequence at least 97.5% identical to SEQ ID NO:8; a 16S-rRNA sequence at least 94.5% identical to SEQ ID NO:9; and a genomic nucleic acid marker having at least 95% local identity to a nucleic acid sequence set forth in any one of SEQ ID NOs:29, 30, 31, 32, and 33 over 90% coverage. Each possibility represents a separate embodiment of the present invention.

According to certain embodiments, the functional homolog of microbial strain S2492 comprises at least one of: a 16S-rRNA sequence at least 85% identical to SEQ ID NO:1; a 16S-rRNA sequence at least 97.5% identical to SEQ ID NO:10; a 16S-rRNA sequence at least 94.5% identical to SEQ ID NO: 11; and a genomic nucleic acid marker having at least 95% local identity to a nucleic acid sequence set forth in any one of SEQ ID NOs:34, 35, 36, 37, and 38 over 90% coverage. Each possibility represents a separate embodiment of the present invention.

According to certain embodiments, the functional homolog of strain S2441 comprises a 16S-rRNA sequence at least 85% identical to SEQ ID NO:2.

According to certain embodiments, the functional homolog of strain S2876 comprises a 16S-rRNA sequence at least 85% identical to SEQ ID NO:3.

According to certain embodiments, the functional homolog of strain S2550 comprises a 16S-rRNA sequence at least 85% identical to SEQ ID NO:4.

According to certain embodiments, the functional homolog comprises a 16S-rRNA sequence at least 85% identical to SEQ ID NOs:5, 6, or 7. Each possibility represents a separate embodiment of the present invention.

According to certain embodiments, the present invention provides an isolated culture of any one of the isolated strains or functional homologs thereof described herein.

According to certain embodiments, the present invention provides an enriched culture of any one of the isolated strains or functional homologs thereof described herein.

According to certain embodiments, the present invention provides a biologically pure culture of any one of the isolated strains or functional homologs thereof described herein.

It is to be explicitly understood that the present invention encompasses a bacterium of the bacterial strains or the functional homolog strains thereof as well as a bacterium derivable from bacterial strains or the functional homolog strains thereof.

According to certain embodiments, the microbial strains and functional homologous thereof of the present invention are characterized by plant growth-promoting activity.

According to certain embodiments, the present invention provides a genus of microorganisms comprising any of the DNA sequences described hereinabove and which is characterized by plant growth-promoting activity, including, but not limited to, enhancing the health, growth and/or yield of a plant, as described herein. In some embodiments, a microbial strain is a Variovorax strain. In some currently exemplary embodiments, a microbial strain is a Variovorax paradoxus strain. In some currently exemplary embodiments, a microbial strain is a S3167 Variovorax paradoxus (NRRL No. B-67735) strain. In other currently exemplary embodiments, a microbial strain is a S2492 Variovorax paradoxus (NRRL No. B-67736) strain.

According to certain embodiments, the present invention provides a microbial preparation comprising at least one microbial strain of the present invention, isolated culture, enriched culture or biologically pure culture thereof, as described hereinabove.

According to certain embodiments, the present invention provides a microbial preparation comprising a combination of at least two of the microbial strains, isolated cultures, enriched cultures or biologically pure cultures thereof as described hereinabove. According to some exemplary embodiments, the combination comprises the isolated Variovorax paradoxus strains S3167 and S2492, isolated cultures, enriched cultures, or biologically pure cultures thereof. According to further exemplary embodiments, the combination comprises the isolated Variovorax paradoxus strain S3167; the isolated Variovorax paradoxus strain S2492; and the isolated Niastella gongjuensis strain S2876; isolated cultures, enriched cultures, or biologically pure cultures thereof. The isolated strains and cultures are as described herein. According to certain exemplary embodiments, the combination of at least two strains forms a synthetic consortium as defined herein.

In some embodiments, the present invention provides a microbial composition comprising at least one microbial strain of the present invention, or an isolated culture, biologically pure culture or enriched culture thereof.

In some embodiments, the present invention provides a microbial composition comprising at least two isolated microbial strains, isolated cultures, biologically pure cultures or enriched cultures thereof, wherein the composition comprises at least one Variovorax strain, and at least one additional microbial strain selected from the group consisting of P0032_C7, P0048_B9, P0050_F5 (also referred to as S2199), P0035_B2 (also referred to as S2145; NRRL Deposit No. B-67091), P0020_B1, P0047_A1 (also referred to as S2284; NRRL Deposit No. B-67102), P0033_E1 (also referred to as S2177), P0032_A8 (also referred to as S2181; NRRL Deposit No. B-67099), P0049_E7, P0042_A8 (also referred to as S2167), P0042_D5 (also referred to as S2165), P0042_B2 (also referred to as S2168; NRRL Deposit No. B-67096), P0042_B12 (also referred to as S2189), P0042_C2 (also referred to as S2173; NRRL Deposit No. B-67098), P0042_D10 (also referred to as S2172; NRRL Deposit No. B-67097), P0044_A3 (also referred to as S2476), P0018_A11, P0044_A5, P0047_E2, P0047_C1, P0038_D2 (also referred to as S2166), P0042_E1, P0047_E8, P0018_A1, P0058_B9 (also referred to as S2159; NRRL Deposit No. B-67092), P0054_E8 (also referred to as S2161; NRRL Deposit No. B-67094), P0054_F4 (also referred to as S2164), P0057_A3 (also referred to as S2160; NRRL Deposit No. B-67093), P0061_E11 (also referred to as S2142), P0019_A12 (also referred to as S2163; NRRL Deposit No. B-67095), P0147_D10 (also referred to as S2291; NRRL Deposit No. B-67104), P0147_G10 (also referred to as S2292; NRRL Deposit No. B-67105), P0160_F7 (also referred to as S2351), P0140_C10 (also referred to as S2300; NRRL Deposit No. B-67107), S2387, P0157_G5 (also referred to as S2303: NRRL Deposit No. B-67108, P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106), S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. B-67100), S2285 (NRRL Deposit No. B-67103), S2477, S2376, S2420, 52424, S2445, S2333, S2329, S2327, S2330, S2423 (NRRL Deposit No. B-67115), 52435, S2158, 52437, S2332, S2521, 52228, S2473, P0156_G2, P0154_G3, S2487, S2488, S2421 (NRRL Deposit No. B-67114), P0105_C5, P0154_H3, P0156_G1, SI112 (NRRL Deposit No. B-67090), S2375 (NRRL Deposit No. B-67110), S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRL Deposit No. B-67445), S2145-2 (NRRL Deposit No. B-67331), S2292-2 (NRRL Deposit No. B-67332), S2300-2 (NRRL Deposit No. B-67333), S2303-2 (NRRL Deposit No. B-67334), S2375-2 (NRRL Deposit No. B-67335), S2382-2 (NRRL Deposit No. B-67336), S2423-2 (NRRL Deposit No. B-67337), S2669-2 (NRRL Deposit No. B-67338), a functional homolog thereof or a strain derived therefrom.

According to certain exemplary embodiments, the Variovorax strain is a Variovorax paradoxus strain. According to certain exemplary embodiments, the Variovorax paradoxus strain is selected from the group consisting of strain S3167 and strain S2492.

According to certain embodiments, the composition comprises at least two, at least three, or at least four additional microbial strains. Other embodiments provide a synthetic microbial consortium comprising a) a first set of microbes comprising one or more microbes that promote plant health, growth, and/or yield; and b) a second set of microbes comprising one or more microbes that increase the competitive fitness of the first set of microbes in a); wherein the first and the second sets of microbes are combined into a single mixture as a synthetic consortium. In some embodiments, the synthetic consortium promotes or enhances plant health, growth and/or yield.

According to certain embodiments, the first and/or the second microbial sets of the synthetic consortium comprises at least one microbial strain of the present invention.

In some embodiments, the present invention provides a microbial composition comprising at least one of the microbial strains, cultures, combinations thereof and synthetic consortia of the present invention. According to certain embodiments, the composition further comprises a plant or a plant seed.

According to some embodiments, the plant or the plant seed comprises at least one genetically modified cell conferring enhancement of at least one trait compared to a non-modified plant or plant seed, wherein the trait is selected from the group consisting of, but not limited to, grain yield, insect control, disease resistance, drought resistance, herbicide resistance and any combination thereof. Each possibility represents a separate embodiment of the present invention.

According to certain embodiments, the composition further comprises at least one agriculturally effective amount of a compound or composition selected from, but not limited to, a nutrient, a fertilizer, an acaricide, a bactericide, a fungicide, an insecticide, a microbicide, a nematicide, a pesticide and combinations thereof. Each possibility represents a separate embodiment of the present invention. In some embodiments of the microbial compositions described herein, the microbial composition further comprises a carrier. According to certain embodiments, the carrier is selected from the group consisting of, but not limited to, an organic or an inorganic carrier and combinations thereof. In some embodiments, the carrier suitable for the microbial compositions is selected from the group consisting of, but not limited to, silt, peat, turf, talc, lignite, kaolinite, pyrophyllite, zeolite, montmorillonite, alginate, press mud, sawdust, vermiculite and combinations thereof. Each possibility represents a separate embodiment of the present invention.

In some embodiments, the carrier is a plant seed. In some embodiments, the microbial composition is prepared as a formulation selected from, but not limited to, an emulsion, a colloid, a dust, a granule, a pellet, a powder, a spray, and a solution. Each possibility represents a separate embodiment of the present invention.

According to certain embodiments, when the carrier is a plant seed, the microbial composition is formulated as a seed coating formulation.

The present invention further provides a plant or a plant seed comprising at least one of the microbial strains, cultures, microbial consortia or a composition comprising same according to the teachings of the present invention.

According to certain embodiments, the plant or the plant seed is coated with the at least one microbial strain, culture thereof, microbial consortium or a composition comprising same.

According to certain embodiments, the present invention provides a plant seed coated with a seed coating formulation comprising at least one microbial strain, microbial culture, or microbial consortium of the present invention.

According to certain embodiments, the plant, part thereof or the plant seed comprises at least one modified cell conferring at least one enhanced trait compared to a non-modified plant, plant part or plant seed, wherein the trait is selected from the group consisting of, but not limited to, grain yield, insect control, disease resistance, drought resistance, herbicide resistance and any combination thereof.

According to yet another aspect, the present invention provides a method for treating a plant seed, the method comprising exposing or contacting the plant seed with at least one microbial strain according to the present embodiments or a culture thereof. In some embodiments, the method comprises exposing or contacting the plant seed with a microbial composition according to the present invention. According to certain embodiments, exposing or contacting the plant seed with the at least one microbial strain, culture thereof or composition comprising same is performed during the process of seed priming.

According to yet additional aspect, the present invention provides a method for enhancing the health, growth and/or yield of a plant, the method comprising applying to the plant or part thereof an effective amount of at least one microbial strain, culture thereof, microbial consortium or a composition comprising same of the invention to the plant, plant part, or plant's surroundings.

According to a further aspect, the present invention provides a method for preventing, inhibiting or ameliorating the development of a plant disease caused by a plant pathogen, the method comprising applying to the plant, part thereof, or the plant growth medium an effective amount of at least one isolated microbial strain, a culture thereof, a microbial preparation or a composition comprising same according to the teachings of the invention.

According to certain embodiments, the plant's surroundings is selected from the group consisting of a plant growth liquid medium and soil. According to certain embodiments, the soil comprises the plant's immediately adjacent soil layer and/or rhizosphere.

In some embodiments, the method comprises growing one or more microbial strains of the invention in the liquid growth medium or soil of a host plant or plant part prior to or concurrent with the host plant's growth in said liquid growth medium or soil.

In some embodiments of the above method, a microbial strain is applied to the plant, plant part, or to the plant's surroundings (e.g., liquid cell medium, immediate soil layer or rhizosphere) in a culture or a composition according to the present embodiments at a concentration that is at least 2×, 5×, 10×, 100×, 500×, or 1000× the concentration of the same microbial strain found in nature or detected in an untreated control plant, plant part, or the control plant's surroundings. In some embodiments, upon or after application, the concentration of the microbial strain in the treated plant, plant part, or the plant's surroundings (e.g., liquid cell medium, immediate soil layer or rhizosphere) is at least 2×, 5×, 10×, 100×, 500×, or 1000× the concentration of the same microbial strain found in nature or detected in an untreated control plant, plant part, or the control plant's surroundings. In some embodiments of the above method, a microbial strain is applied to the plant, plant part, or to the plant's surroundings (e.g., liquid cell medium, immediate soil layer or rhizosphere) in a culture or a composition at a concentration of at least 1×10² CFU/mL. In some embodiments, concentration ranges are from about 1×10² to about 1×10¹⁰ CFU/mL, typically at concentrations ranging from 1×10⁵ to 1×10⁹ CFU/mL. In some embodiments, application of a microbial strain (PGPM) as described herein to a plant, plant part, or to the plant's surroundings in a culture or a composition at a concentration that is at least 1×10⁶ CFU/mL leads to a concentration of the microbial strain in the treated plant, plant part or the plant's surroundings that is at least 2× the amount of the strain found in an untreated plant or its surroundings.

Other embodiments provide a non-naturally occurring plant. In some embodiments, the non-naturally occurring plant is artificially infected with one or more microbial strains (PGPMs) according to the present embodiments. Further provided in some embodiments of this aspect is a plant seed, reproductive tissue, vegetative tissue, regenerative tissue, plant part or progeny of a plant.

It is to be understood that any combination of each of the aspects and the embodiments disclosed herein is explicitly encompassed within the disclosure of the present invention.

Other objects, features and advantages of the present invention will become clear from the following description.

### BRIEF DESCRIPTION OF THE DRAWINGS

FIG. 1 shows relative abundances of consortium strains detected in positive control (consortium only) samples, showing specificity by the selected reporter region. For the V5V6 region of 16S rDNA, the two Variovorax strains are represented by a single tag. For the V1V8 region, the two Variovorax strains are represented by different tags, and unique operons within Niastella are resolved. The relative abundance of V1V8 tags was normalized by operon copy number.

FIG. 2 shows HiSeq V5V6 results for relative abundances of microbes in root ball powder as revealed by sequencing trials.

FIG. 3 shows Loop V1V8 results for relative abundances of microbes in root ball powder as revealed by sequencing trials, where Variovorax paradoxus strains S2492, B-67736 and S3167, B-67735 are separated to two tags.

### DETAILED DESCRIPTION OF THE INVENTION

The present invention provides microbial strains, particularly bacterial strains, characterized as plant growth-promoting microbes (PGPMs). The plant growth-promoting bacterial strains of the invention have been isolated from soil samples collected from the root or the root area of plants showing over-performance with regard to size (height and weight) and yield. Thus, advantageously, the microbial strains of the present invention are thought to be directly associated with enhancement of traits having significant importance for agricultural crop plants.

### Definitions

Unless otherwise defined, all terms of art, notations and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed by those skilled in the art.

The singular form “a”, “an”, and “the” include plural references unless the context clearly dictates otherwise. For example, the term “a cell” includes one or more cells, including mixtures thereof.

As used herein, an isolated strain of a microbe is a strain that has been removed from its natural milieu. As such, the term “isolated”, with reference to the microbial strain or its culture, does not necessarily reflect the extent to which the microbe has been purified. However, according to certain embodiments, an “isolated” culture has been purified at least 2× or 5× or 10× or 50× or 100× from the raw material from which it is isolated. As a non-limiting example, if a culture is isolated from soil as raw material, the organism can be isolated to an extent that its concentration in a given quantity of purified or partially purified material (e.g., soil) is at least 2× or 5× or 1× or 50× or 100× of that in the original raw material.

A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants as well as undesirable chemical contaminants.

As used herein, a “biologically pure” strain is intended to mean a strain separated from materials with which it is normally associated in nature. A strain associated with other strains, or with compounds or materials that it is not normally found with in nature, is still defined as “biologically pure”. A monoculture of a particular strain is, of course, “biologically pure”. In different embodiments, a “biologically pure” culture has been purified at least 2× or 5× or 10× or 50× or 100× or 1000× or higher (to the extent considered feasible by a skilled person in the art) from the material with which it is normally associated in nature. As a non-limiting example, if a culture is normally associated with soil, the organism can be biologically pure to an extent that its concentration in a given quantity of purified or partially purified material with which it is normally associated (e.g. soil) is at least 2× or 5× or 10× or 50× or 100×, or 1000× or higher (to the extent considered feasible by a skilled person in the art) than that in the original unpurified material.

As used herein, the term “enriched culture” of an isolated microbial strain refers to a microbial culture wherein the total microbial population of the culture contains more than 50%, 60%, 70%, 80%, 90%, or 95% of the isolated strain.

The term “culturing”, as used herein, refers to the propagation of organisms on or in media of various kinds. Suitable media are known to a person with ordinary skill in the art.

The term “composition” as used herein refers to a combination of an active agent (e.g., a PGPM or microbial strain described herein) and at least one other compound, carrier, or composition, which can be inert (for example, a detectable agent or label or liquid carrier) or active, such as, but not limited to, a fertilizer, nutrient, or pesticide. A microbial composition refers to a composition comprising at least one microbial species.

Ribosomes, which are comprised of numerous ribosomal proteins and three ribosomal RNA (rRNA) molecules, are a key component of protein synthesis. The 16S subunit rRNA, which is encoded by the 16S rRNA gene, has been the focus of much attention in microbial phylogenetic studies. The 16S rRNA gene sequence is highly conserved between taxonomic groups, yet also possesses regions that are highly polymorphic. Moreover, the rate of change in the RNA sequence is thought to have been relatively constant over evolutionary time, enabling scientists to determine the relative relatedness of different organisms based on their 16S rRNA sequences or parts thereof.

The term “effective amount,” as used herein, is an amount sufficient to effect beneficial and/or desired results. An effective amount can be administered in one or more administrations. In terms of treatment, inhibition or protection, an effective amount is that amount sufficient to ameliorate, stabilize, reverse, slow or delay progression of a target infection, abiotic stress, or disease state. The expression “effective microorganism” used herein in reference to a microorganism is intended to mean that the subject strain exhibits a degree of promotion of plant health, growth and/or yield, or, in certain embodiments, a degree of inhibition of a pathogenic disease that exceeds, at a statistically significant level, that of an untreated control. In some instances, the expression “an effective amount” is used herein in reference to that quantity of microbial treatment which is necessary to obtain a beneficial or desired result relative to that occurring in an untreated control under suitable conditions of treatment as described herein. For example, the expression “an agriculturally effective amount” is used herein in reference to that quantity of microbial treatment which is necessary to obtain an agriculturally beneficial or desired result relative to that occurring in an untreated control under suitable conditions of treatment as are known in the art and as described herein. The effective amount of an agricultural formulation or composition that should be applied for the improvement of plant health, growth and/or yield, for the control of, e.g., insects, plant diseases, or weeds, can be readily determined via a combination of general knowledge of the applicable field.

The term “nutrient” as used herein refers to a compound or composition that is able to provide one or more nutrient elements to plants. In some embodiments, a nutrient provides one or more nutrient elements selected from nitrogen (N), phosphorus (P), potassium (K), calcium (Ca), magnesium (Mg), sulfur (S), iron (Fe), manganese (Mn), zinc (Zn), copper (Cu), nickel (Ni), boron (B) and molybdenum (Mo) to the plants. In some embodiments, a nutrient as used herein provides at least one of nitrogen (N), phosphorus (P) and potassium (K) to the plants. In some embodiments, a nutrient provides at least one of calcium (Ca), magnesium (Mg) and sulfur (S) to the plants. In some embodiments, a nutrient of embodiments of the present invention provides at least one of iron (Fe), manganese (Mn), zinc (Zn), copper (Cu), nickel (Ni), boron (B) and molybdenum (Mo) to the plants. In some embodiments, a nutrient is a compound or composition that promotes the plant uptake of one or more nutrient elements selected from nitrogen (N), phosphorus (P), potassium (K), calcium (Ca), magnesium (Mg), sulfur (S), iron (Fe), manganese (Mn), zinc (Zn), copper (Cu), nickel (Ni), boron (B) and molybdenum (Mo), from the soil.

The term “fertilizer” as used herein refers to a compound or composition that is added to plants or soil to improve plant health, growth and/or yield. In some embodiments, a fertilizer improves plant health, growth and/or yield by providing a nutrient (such as the ones described hereinabove) to the plant. Fertilizers include, but are not limited to, inorganic fertilizers, organic (or natural) fertilizers, granular fertilizers and liquid fertilizers. Granular fertilizers are solid granules, while liquid fertilizers are made from water soluble powders or liquid concentrates that mix with water to form a liquid fertilizer solution. In some embodiments, plants can quickly take up most water-soluble fertilizers, while granular fertilizers may need a while to dissolve or decompose before plants can access their nutrients. High-tech granular fertilizers have “slow-release,” “timed-release,” or “controlled-release” properties, synonymous terms meaning that they release their nutrients slowly over a period of time. Organic fertilizer may come from organic sources such as, but not limited to, compost, manure, blood meal, cottonseed meal, feather meal, crab meal, or others, as opposed to synthetic sources. There are also some natural fertilizers that are not organic, such as Greensand, which contain potassium, iron, calcium, and other nutrients. Organic fertilizers depend on the microbes in the soil to break them down into digestible bits for plants. Inorganic fertilizers are also known as synthetic or artificial fertilizers. Inorganic fertilizers are manufactured.

A “bacteriostatic” compound or agent, or a bacteriostat (sometimes abbreviated to Bstatic), is a biological or chemical agent that stops bacteria from growing and reproducing, while not necessarily harming them otherwise. An “acaricide” means a compound or composition that increases the mortality of, or materially inhibits the growth, reproduction, or spread of undesired acarids, including but not limited to dust mites. A “bactericide” means a compound or composition that increases the mortality of, or materially inhibits the growth, reproduction, or spread of undesired bacteria, such as (but not limited to) those unfavorable for the plant growth. A “fungicide” refers to a compound or composition that increases the mortality of, or materially inhibits the growth, reproduction, or spread of undesired fungi, such as (but not limited to) those unfavorable for the plant growth. A “nematicide” refers to a compound or composition that increases the mortality of, or materially inhibits the growth, reproduction, or spread of undesired nematodes. An “insecticide” refers to a compound or composition that increases the mortality of, or materially inhibits the growth, reproduction, or spread of undesired insects, such as (but not limited to) those that are harmful for the plant growth. A “microbicide” refers to a compound or composition that increases the mortality of, or materially inhibits the growth, reproduction, or spread of undesired microbes, such as (but not limited to) those that are harmful for the plant growth. A “pesticide” refers to a compound or composition that increases the mortality of, or materially inhibits the growth of, materially inhibits the reproduction of, or materially inhibits the spread of undesired pests, such as (but not limited to) those that are harmful for the plant growth.

A “carrier” as used herein refers to a substance or a composition that support the survival of the microbes. Such carriers may be either organic or non-organic. In some embodiments, a carrier may be an agriculturally accepted carrier.

“Seed priming” or “priming of seed” means controlling the hydration level within seeds so that the metabolic activity necessary for germination can occur but elongation by the embryonic axis, i.e. usually radicle emergence, is prevented. Different physiological activities within the seed occur at different moisture levels (Leopold and Vertucci, 1989, Moisture as a regulator of physiological reactions in seeds. In: Seed Moisture, eds. P. C. Stanwood and M. B. McDonald. CSSA Special Publication Number 14. Madison, Wis.: Crop Science Society of America, pp. 51-69; Taylor, 1997, Seed storage, germination and quality. In: The Physiology of Vegetable Crops, ed. H. C. Wien. Wallingford, U.K.: CAB International, pp. 1-36). The last physiological activity in the germination process is radicle emergence. The initiation of radicle emergence requires a high seed water content. By limiting seed water content, all the metabolic steps necessary for germination can occur without the irreversible act of radicle emergence. Prior to radicle emergence, the seed is considered desiccation tolerant, thus the primed seed moisture content can be decreased by drying. After drying, primed seeds can be stored until time of sowing. For example, in some embodiments, a plant seed can be exposed to or placed in contact with a microbial strain or a culture thereof, or a composition according to embodiments of the present invention during the hydration treatment of seed priming. In some embodiments, the exposure or contact of a plant seed with the microbial strain or a culture thereof or a composition of the present invention during the priming process improves seed germination performance, as well as later plant health, plant growth, and/or final plant yield.

As used herein, the term “endophyte” refers to an endosymbiont that lives within a plant for at least part of its life. Endophytes may be transmitted either vertically (directly from parent to offspring) or horizontally (from individual to unrelated individual). For example, vertically-transmitted fungal endophytes are asexual and transmit from the maternal plant to offspring via fungal hyphae penetrating the host's seeds. Bacterial endophytes can also be transferred vertically from seeds to seedlings (Ferreira et al., FEMS Microbiol. Lett. 287:8-14, 2008). In some embodiments, horizontally-transmitted endophytes are typically sexual, and transmitted via spores that can be spread by wind and/or insect vectors. Microbial endophytes of crop plants have received considerable attention with respect to their ability to control disease and insect infestation, as well as their potential to promoting plant growth. For instance, some microbial strains described herein may be able to establish as endophytes in plants that come in contact with them. Such microbial strains are microbial endophytes.

The term “pathogen” as used herein refers to an organism such as an alga, an arachnid, a bacterium, a fungus, an insect, a nematode, a parasitic plant, a protozoan, a yeast, or a virus capable of producing a disease in a plant or animal. The term “phytopathogen” as used herein refers to a pathogenic organism that infects a plant. A “pathogenic disease” is a disease, such as a plant disease, that is caused by at least one pathogen. A “phytopathogenic disease” is a disease, such as a plant disease, that is caused by at least one phytopathogen. Some pathogens that may cause plant pathogenic diseases include, but are not limited to, Colletotrichum, Fusarium, Gibberella, Monographella, Penicillium, and Stagnospora organisms.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are well-known to those of skill in the art, for instance, using publicly available computer software such as BLAST, software of the National Center of Biotechnology Information (NCBI). Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

As used herein in reference to a nucleic acid and polypeptide, the term “variant” denotes a polypeptide, protein or polynucleotide molecule with some differences, generated synthetically or naturally, in their amino acid or nucleic acid sequences as compared to a reference polypeptide or polynucleotide, respectively. For example, these differences include substitutions, insertions, deletions or any desired combinations of such changes in a reference polypeptide or polypeptide. Polypeptide and protein variants can further consist of changes in charge and/or post-translational modifications (such as glycosylation, methylation. phosphorylation, etc.).

The terms “variant”, “functional variant”, “functional homolog” and “functionally homologous” when used herein in reference to a microorganism, interchangeably refer to a microbial strain having identifying characteristics of the species to which it belongs, while having at least one nucleotide sequence variation or identifiably different trait with respect to the parental strain. The sequence variation or different trait results in a microbial strain that is endowed with substantially the same ensemble of biological activities, particularly promoting plant health, growth and or yield (about 10%, 20%, 40%, 50%, or 60% enhancement when tested under the same conditions) as that of the strain of the invention, where the trait is genetically based (heritable).

The term “PGPM” refers to plant-growth promoting microorganisms (or microbes). In some embodiments, PGPMs not only can promote plant health, growth and/or yield, but also can survive and multiply in microhabitats associated with the root surface, in competition with other microbiota, and/or are able to colonize the root, at least for the time needed to express their plant promotion and/or protection activities. In some embodiments, microbial strains whose 16S rRNA gene comprises a nucleic acid sequence selected from the SEQ ID NOs.:1-11, and variants or progeny thereof, are PGPMs. According to certain embodiments, a PGPM is a Variovorax strain. In some embodiments, a PGPM is a Variovorax paradoxus strain. In some exemplary embodiments, the PGPM is a microbial strain selected from the group consisting of a S3167 Variovorax paradoxus strain (NRRL No. B-67735), a S2492 Variovorax paradoxus strain (NRRL No. B-67736), a strain derived from S3167 Variovorax paradoxus (NRRL No. B-67735) and a strain derived from S2492 Variovorax paradoxus (NRRL No. B-67736).

Strains NRRL No. B-67735 and B-67736 were deposited with the Agricultural Research Service Culture Collection (NRRL) International Depositary Authority, 1815 N. University Street Peoria, Ill. 61604 U.S.A., on Feb. 4, 2019 and received deposit numbers of Feb. 19, 2019.

The PGPMs, isolates, cultures, compositions or synthetic consortia promote or enhance plant health, growth or yield, and/or have plant growth-promoting activity. The term “plant growth-promoting activity”, as used herein, encompasses a wide range of improved plant properties, including, for example without limitation, improved nitrogen fixation, improved root development, increased leaf area, increased plant yield, increased seed germination, increased photosynthesis, or an increase in accumulated biomass of the plant. In some embodiments, the microbial strains, isolates, cultures, compositions or synthetic consortia as described herein improves stress tolerance (e.g., tolerance to drought, flood, salinity, heat, pest), improves nutrient uptake, plant heath and vigor, improves root development, increases leaf area, increases plant yield, increases seed germination, or promotes an increase in accumulated biomass of the plant. In some embodiments, the microbial strains, isolates, cultures, compositions or synthetic consortia as described herein increase the size or mass of a plant or parts thereof, as compared to a control plant, or parts thereof or as compared to a predetermined standard. In some embodiments, the microbial strains, isolates, cultures, compositions or synthetic consortia as described herein promote plant growth by promoting seed germination, as compared to a control seed. In some embodiments, the microbial strains, isolates, cultures, compositions or synthetic consortia as described herein improve the health, vigor, and/or yield of a plant, as compared to a control plant.

As used herein, the term “yield” or “grain yield” refers to the amount of harvestable plant material or plant-derived product and is normally defined as the measurable produce of economic value of a crop. For crop plants, yield also means the amount of harvested material per acre or unit of production. Yield may be defined in terms of quantity or quality. The harvested material may vary from crop to crop, for example, it may be seeds, above ground biomass, roots, fruit, cotton fibers, any other part of the plant, or any plant-derived product which is of economic value. The term yield also encompasses yield potential, which is the maximum obtainable yield. Yield may be dependent on a number of yield components, which may be monitored by certain parameters. These parameters are well known to persons skilled in the art and vary from crop to crop. The term yield also encompasses harvest index, which is the ratio between the harvested biomass over the total amount of biomass.

In some embodiments, the microbial strains, isolates, cultures and compositions, including compositions comprising a plant or plant seed, induce a yield improvement that is at least 2% increase, at least 3% increase, at least 4% increase, at least 5% increase, at least 10% increase, at least 15% increase, at least 20%, at least 25% increase, at least 50% increase, at least 75% increase, or at least a 100% increase in the property being measured compared to a control plant. Thus, as non-limiting examples, the microbial strains, isolates, cultures and compositions according to embodiments of the present invention may produce an above stated percentage increase in nitrogen fixation, or an above stated increase in total root weight, or in leaf area or in plant product yield (e.g., an above stated percentage increase in plant product weight), or an increased percentage of seeds that germinate within 10 days or 14 days or 30 days, or rate of photosynthesis (e.g., determined by CO₂ consumption) or accumulated biomass of the plant (e.g., determined by weight and/or height of the plant). According to certain exemplary embodiments, the plant produce is—a food item produced by the plant.

According to some embodiments, the plant or the plant seed comprises at least one modified cell conferring at least one enhanced trait compared to a non-modified plant.

The term “control plant”, as used herein, provides a reference point for measuring changes in phenotype of the subject plant, and may be any suitable plant cell, seed, plant component, plant tissue, plant organ or whole plant. A control plant may comprise, for example (but not limited to), (a) a wild-type plant or cell, i.e., of the same genotype as the starting material for the genetic alteration which resulted in the subject plant or cell; (b) a plant or cell of the genotype as the starting material but which has been transformed with a null construct (i.e., a construct which has no known effect on the trait of interest, such as a construct comprising a reporter gene); (c) a plant or cell which is a non-transformed segregant among progeny of a subject plant or cell; (d) a plant or cell which is genetically identical to the subject plant or cell but which is not exposed to the same treatment (e.g., inoculant treatment) as the subject plant or cell; (e) the subject plant or cell itself, under conditions in which the gene of interest is not expressed; or (f) the subject plant or cell itself, under conditions in which it has not been exposed to a particular treatment such as, for example, an inoculant or combination of inoculants, microbial strains, and/or other chemicals.

The term “inoculant” as used herein refers to any culture or preparation that comprises at least one microorganism. In some embodiments, an inoculant (sometimes as microbial inoculant, or soil inoculant) is an agricultural amendment that uses beneficial microbes, such as PGPMs, (including, but not limited to endophytes) to promote plant health, growth and/or yield. Many of the microbes suitable for use in an inoculant may form symbiotic relationships with the target crops where both parties benefit (mutualism).

The term “competitive fitness” as used herein refers to the fitness of the microbes to compete with their neighbors for space and resources. Fitness means the ability or propensity of a given genotype (e.g., a 16S rRNA gene sequence) to both survive and reproduce in a given environment.

The term “biofertilizers” as used herein designate the biological products which contain microorganisms providing direct and/or indirect gains in plant health, growth and/or yield.

The term “bioreactor” as used herein refers to any device or system that supports a biologically active environment. As described herein a bioreactor is a vessel in which microorganisms including the microorganism of the embodiments of this application can be grown.

Diverse plant-associated microorganisms, including, but not limited to, many rhizobacterial species, can positively impact plant health and physiology in a variety of ways. These beneficial microbes are generally referred to as PGPMs, such as plant growth-promoting bacteria (PGPB) or plant growth-promoting rhizosphere (PGPR). Isolated strains of microorganisms have been reported to have plant growth-promoting activity and/or biocontrol activity, and new genera and species with similar activities are still being discovered. Additionally, within some bacterial genera, multiple species and subspecies of biocontrol agents have been identified and can be found across multiple spatial scales, from the global level to farm level, and even on single plants. Furthermore, it has been reported that some individual microbial isolates may display biocontrol and/or plant growth-promoting activity not only on the plants or crops from which they were obtained but also on other crops. This indicates the universal nature of some genotypes, especially those with a wide geographic distribution. If introduced in sufficient numbers and kept active for a sufficient duration, a single microbial population can have a significant impact on plant health.

The present invention discloses new microbial strains that are characterized as PGPMs. According to certain embodiments, the microbial strain or functional homolog thereof interacting with the host plant is present in the plant habitat, particularly in the rhizosphere (soil around root).

According to one aspect, the present invention provides an isolated microbial strain or a functional homolog thereof, wherein the isolated microbial strain is selected from the group consisting of:
(1) strain S3167, the strain being selected from the group consisting of:
   a. a strain deposited under Accession Number NRRL No. B-67735;
   b. a strain comprising a 16S-rRNA sequence comprising a nucleic acid sequence set forth in any one of SEQ ID NOs:1, 8, 9 or any combination thereof; and
   c. a strain comprising at least one genomic marker comprising the nucleic acid sequence set forth in any one of SEQ ID NOs:29, 30, 31, 32, and 33;
(2) strain S2492, the strain being selected from the group consisting of:
   a. a strain deposited under Accession Number NRRL No. B-67736;
   b. a strain comprising a 16S-rRNA sequence comprising a nucleic acid sequence set forth in any one of SEQ ID NOs:1, 10, 11, or any combination thereof; and
   c. a strain comprising at least one genomic marker comprising the nucleic acid sequence set forth in any one of SEQ ID NOs:34, 35, 36, 37, and 38;
(3) strain S2441, the strain comprising a 16S-rRNA sequence comprising the nucleic acid sequence set forth in SEQ ID NO:2;
(4) strain S2876, the strain comprising a 16S-rRNA sequence comprising the nucleic acid sequence set forth in SEQ ID NO:3;
(5) strain S2550, the strain comprising a 16S-rRNA sequence comprising the nucleic acid sequence set forth in SEQ ID NO:4; and
(6) a strain comprising a 16S-rRNA sequence comprising a nucleic acid sequence set forth in any one of SEQ ID NOs:5, 6, or 7.

Each possibility represents a separate embodiment of the present invention.

According to certain embodiments, the functional homolog of microbial strain S3167 comprises at least one of: a 16S-rRNA sequence at least 85% identical to SEQ ID NO:1; a 16S-rRNA sequence at least 97.5%; at least 97.6%, at least 97.7%, at least 97.8%, at least 97.9%, at least 98%, at least 98.5%, at least 98.6%, at least 98.7%, at least 98.8%, at least 98.9%, at least 99%, at least 99.5%, at least 99.6%, at least 99.7%, at least 99.8%, at least 99.9%, or 100% identical to SEQ ID NO:8; a 16S-rRNA sequence at least 94.5%, at least 94.6%, at least 94.7%, at least 94.8%, at least 94.9%, at least 95%, at least 96%, at least 97%, at least 98%, at least 99% or 100% identical to SEQ ID NO:9; and a genomic nucleic acid marker having at least 95%, at least 96%, at least 97%, at least 98%, at least 99% or 100% local identity to a nucleic acid sequence set forth in any one of SEQ ID NOs:29, 30, 31, 32, and 33 over 90°/% coverage. Each possibility represents a separate embodiment of the present invention.

According to certain embodiments, the functional homolog of microbial strain S2492 comprises at least one of: a 16S-rRNA sequence at least 85% identical to SEQ ID NO:1; a 16S-rRNA sequence at least 97.5% identical to SEQ ID NO:10; a 16S-rRNA sequence at least 94.5% at least 97.6%, at least 97.7%, at least 97.8%, at least 97.9%, at least 98%, at least 98.5%, at least 98.6%, at least 98.7%, at least 98.8%, at least 98.9%, at least 99%, at least 99.5%, at least 99.6%, at least 99.7%, at least 99.8%, at least 99.9%, or 100% identical to SEQ ID NO:11; and a genomic nucleic acid marker having at least 95%, at least 95%, least 96%, at least 97%, at least 98%, at least 99% or 100% local identity to a nucleic acid sequence set forth in any one of SEQ ID NOs:34, 35, 36, 37, and 38 over 90% coverage. Each possibility represents a separate embodiment of the present invention.

According to certain embodiments, the functional homolog of strain S2441 comprises a 16S-rRNA sequence at least 85%, at least 86%, at least 87%, at least 88%, at least 89%, at least 90%, at least 91%, at least 92%, at least 93%, at least 94%, at least 95%, least 96%, at least 97%, at least 98%, at least 99/o or 100% identical to SEQ ID NO:2.

According to certain embodiments, the functional homolog of strain S2876 comprises a 16S-rRNA sequence at least 85%, at least 86%, at least 87%, at least 88%, at least 89%, at least 90%, at least 91%, at least 92%, at least 93%, at least 94%, at least 95%, least 96%, at least 97%, at least 98%, at least 99% or 100% identical to SEQ ID NO:3.

According to certain embodiments, the functional homolog of strain S2550 comprises a 16S-rRNA sequence at least 85%, at least 86%, at least 87%, at least 88%, at least 89%, at least 90%, at least 91%, at least 92%, at least 93%, at least 94%, at least 95%, least 96%, at least 97%, at least 98%, at least 99% or 100% identical to SEQ ID NO:4.

According to certain embodiments, the functional homolog comprises a 16S-rRNA sequence at least 85%, at least 86%, at least 87%, at least 88%, at least 89%, at least 90%, at least 91%, at least 92%, at least 93%, at least 94%, at least 95%, least 96%, at least 97%, at least 98%, at least 99/o or 100% identical to SEQ ID NO:5, SEQ ID NO:6, or SEQ ID NO:7. Each possibility represents a separate embodiment of the present invention.

According to certain additional or alternative embodiments, the genomic nucleic acid sequences of the isolated microbial strain and the functional homolog (variant) thereof comprises at least one marker.

As used herein, the term “marker” in relation to a microbe, particularly bacterium, genome, refers to a sub-genomic sequence. The terms “marker” and “sub-genomic sequence” are used herein interchangeably.

According to certain exemplary embodiments, identity of a marker sequence is defined as at least 90% query coverage with at least 95% identity, such as further described herein.

According to certain embodiments, the microbial strain of the present invention or the functional homolog thereof comprises at least two markers, at least three markers, at least four markers, or at least five markers.

According to certain exemplary embodiments, the functional homolog of strain S3167 of the invention comprises at least two markers selected from the group consisting of a marker having a nucleic acid sequence at least about 95%, at least about 95.5%, at least about 96%, at least about 96.5%, at least about 97%, at least about 97.1%, at least about 97.2%, at least about 97.3%, at least about 97.4%, at least about 97.5%, at least about 97.6%, at least about 97. %, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical, to any one of SEQ ID NOs:29-33. Each possibility represents a separate embodiment of the present invention.

According to certain further exemplary embodiments, the functional homolog of strain S2492 of the invention comprises at least two markers selected from the group consisting of a marker having a nucleic acid sequence at least about 95%, at least about 95.5%, at least about 96%, at least about 96.5%, at least about 97%, at least about 97.1%, at least about 97.2%, at least about 97.3%, at least about 97.4%, at least about 97.5%, at least about 97.6%, at least about 97. %, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical, to any one of SEQ ID NOs:34-38. Each possibility represents a separate embodiment of the present invention.

Some embodiments provide a genus of plant growth-promoting microorganisms comprising any of the DNA sequences described herein and which enhances the health, growth and/or yield of a plant, as described herein.

In some embodiments, a microbial strain is selected from the group consisting of S3167 (NRRL Deposit No. B-67735), S2492 (NRRL Deposit No. B-67736), S2441, S2876 (NRRL Deposit No. B-67448), S2550, P0032_C7, P0048_B9, P0050_F5 (also referred to as S2199), P0035_B2 (also referred to as S2145; NRRL Deposit No. B-67091), P0020_B1, P0047_A1 (also referred to as S2284; NRRL Deposit No. B-67102), P0033_E1 (also referred to as S2177), P0032_A8 (also referred to as S2181; NRRL Deposit No. B-67099), P0049_E7, P0042_A8 (also referred to as S2167), P0042_D5 (also referred to as S2165), P0042_B2 (also referred to as S2168; NRRL Deposit No. B-67096), P0042_B12 (also referred to as S2189), P0042_C2 (also referred to as S2173; NRRL Deposit No. B-67098), P0042_D10 (also referred to as S2172; NRRL Deposit No. B-67097), P0044_A3 (also referred to as S2476), P0018_A11, P0044_A5, P0047_E2, P0047_C1, P0038_D2 (also referred to as S2166), P0042_E1, P0047_E8, P0018_A1, P0058_B9 (also referred to as S2159; NRRL Deposit No. B-67092), P0054_E8 (also referred to as S2161; NRRL Deposit No. B-67094), P0054_F4 (also referred to as S2164), P0057_A3 (also referred to as S2160; NRRL Deposit No. B-67093), P0061_E11 (also referred to as S2142), P0019_A12 (also referred to as S2163; NRRL Deposit No. B-67095), P0147_D10 (also referred to as S2291; NRRL Deposit No. B-67104), P0147_G10 (also referred to as S2292; NRRL Deposit No. B-67105), P0160_F7 (also referred to as S2351), P0140_C10 (also referred to as S2300; NRRL Deposit No. B-67107), S2387, P0157_G5 (also referred to as S2303; NRRL Deposit No. B-67108, P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106), S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. B-67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), S2669 (NRRL Deposit No. B-67117), S2651, S2652, S2653, S2654, S2655, S2656, 52668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRL Deposit No. B-67445), S2145-2 (NRRL Deposit No. B-67331), S2292-2 (NRRL Deposit No. B-67332), S2300-2 (NRRL Deposit No. B-67333), S2303-2 (NRRL Deposit No. B-67334), S2375-2 (NRRL Deposit No. B-67335), S2382-2 (NRRL Deposit No. B-67336), S2423-2 (NRRL Deposit No. B-67337), S2669-2 (NRRL Deposit No. B-67338), or a strain derived from any one of these strains.

The microbial strain deposits were made under the provisions of the Budapest Treaty on the International Recognition of the Deposit of Microorganisms for the Purposes of Patent Procedure. Furthermore, these deposits will be maintained under the terms of the Budapest Treaty on the International Recognition of the Deposit of Microorganisms for the Purposes of Patent Procedure. Access to these deposits will be available during the pendency of the application to the Commissioner of Patents and Trademarks and persons determined by the Commissioner to be entitled thereto upon request. Upon allowance of any claims in the application, the Applicant will make available to the public, pursuant to 37 C.F.R. § 1.808, sample(s) of the deposits. The deposits will be maintained in the NRRL depository, which is a public depository, for a period of 30 years, or 5 years after the most recent request, or for the enforceable life of the patent, whichever is longer, and will be replaced if it becomes nonviable during that period.

Some embodiments also provide isolates and cultures of the microbial strains as described herein, and compositions and synthetic consortia comprising various combinations of those microbial strains, isolates or cultures and a plant or plant seed.

In some embodiments, the PGPMs, when applied to seed, plant surfaces, plant parts, or soil, colonizes rhizosphere and/or the interior of the plant and promotes growth of the host plant. In some embodiments, the PGPMs are biofertilizers. In some embodiments, the PGPMs are microbial fertilizers, which supply the plant with nutrients and thereby can promote plant growth in the absence of pathogen pressure. In some embodiments, the PGPMs may directly promote plant growth and/yield through a mechanism selected from the group consisting of, but not limited to, ability to produce or change the concentration of plant hormones; asymbiotic nitrogen fixation; and solubilization of mineral phosphate and/or other nutrients.

In some embodiments, the PGPMs of the invention may affect the plant growth and development as phytostimulators. For example, some PGPMs described herein may have the ability to produce or change the concentration of plant hormones, including, but not limited to the five classical phytohormones, i.e., auxin, ethylene, abscisic acid, cytokinin, and gibberellin. Some PGPMs may also produce enzymes or secondary metabolites that affect phytohormone production in plants. In some embodiments, the PGPMs may have the ability to produce or change the concentration of other hormones as well as certain volatile organic compounds (VOCs) and the cofactor pyrrolquinoline quinone (PQQ), thereby stimulating plant growth and/or yield.

In some embodiments, PGPMs may affect the plant growth and development by modifying nutrient availability or uptake. The PGPMs may alter nutrient uptake rates, for example, by direct effects on roots, by effects on the environment which in turn modify root behavior, and by competing directly for nutrients. Some factors by which PGPMs described herein may play a role in modifying the nutrient use efficiency in soils include, for example, root geometry, nutrient solubility, nutrient availability by producing plant congenial ion form, partitioning of the nutrients in plant and utilization efficiency. For example, a low level of soluble phosphate can limit the growth of plants. Some plant growth-promoting microbes are capable of solubilizing phosphate from either organic or inorganic bound phosphates, thereby facilitating plant growth.

In some embodiments, PGPMs may affect the plant growth and development as plant stress controllers. For example, some PGPMs may control and/or reduce several types of plant stress, including, but not limited to, stress from the effects of phytopathogenic bacteria, stress from polyaromatic hydrocarbons, stress from heavy metal such as Ca²⁺ and Ni²⁺, and stress from salt and severe weather conditions (e.g., drought or flood).

In some embodiments, PGPMs may promote plant health, growth and/or yield directly by controlling phytopathogens or pests in plants. In some embodiments, PGPMs described herein exhibit one or more mechanisms of biological disease control, most of which involve competition and production of metabolites that affect the pathogen directly. Examples of such metabolites include antibiotics, cell wall-degrading enzymes, siderophores, and hydrogen cyanide (HCN). Different mechanisms may be found in a single PGPM strain and act simultaneously. In some embodiments, PGPMs may affect the plant growth and development by producing extracellular siderophores. Some PGPMs described herein may secrete low molecular weight, high affinity ferric-chelating microbial cofactors that specifically enhance their acquisition of iron by binding to membrane bound siderophore receptors. Siderophores are small, high-affinity chelators that bind Fe, making it more (or less) available to certain member of natural microflora. For example, a siderophore may make Fe more available to a plant or microbe that possesses the ability to recognize and import the specific siderophore molecular structure. Many different siderophore types and structures exist with different Fe-binding affinities. Furthermore, exchange of Fe from a siderophore with low Fe-binding affinity to one with higher Fe-binding affinity is known to occur which may further influence Fe availability to any given organism. One of the siderophores produced by some Pseudomonad PGPMs is known as pseudobactin that inhibits the growth of Erwinia cartovora (causal organism for soft-rot of potato) (see, e.g., Kloepper et al. Current Microbiol. 4: 317-320, 1980). Additions of pseudobactin to the growth medium inhibited soft-rot infection and also reduced the number of pathogenic fungi in the potato plant along with a significant increase in potato yield. Most evidence to support the siderophore theory of biological control by PGPM comes from work with the pyoverdines, one class of sideophores that comprises the fluorescent pigments of fluorescent Pseudomonads (Demange et al. in: Iron Transport in Microbes, Plants and Animals, G. Winkelmann, D van der Helm and J B Neilands, eds., ISBN 3 527 26685 2, pp 167-187, 1987). According to the siderophore theory, pyoverdines demonstrate certain functional strain specificity which is due to selective recognition of outer membrane siderophore receptors (Bakker et al. Soil Biology and Biochemistry 19: 443-450, 1989). Production of siderophore(s) may modulate the fitness and/or growth of other strains. In addition to inhibiting certain strains (e.g., Erwinia), production of siderophore(s) can also support the fitness/growth of other microbial strains that possess receptors for a given siderophore but are unable to synthesize the molecule themselves.

In some embodiments, the PGPMs may act indirectly on the plant by increasing the competitive fitness of a second microbial strain (e.g., another PGPM) by, e.g., providing nutrients, metabolites and/or siderophores (and/or by any other benefiting mechanism as described herein) to the second microbial strain. In some embodiments, the PGPMs may act indirectly on the plant by increasing the competitive fitness of a second microbial strain (e.g., another PGPM) by, e.g., providing nutrients, metabolites and/or siderophores (and/or by any other benefiting mechanism as described herein) to the second microbial strain, and/or by decreasing the competitive fitness of a third microbial strain that inhibits, competes with, or excludes or otherwise has a negative impact on the fitness of the second microbial strain.

In some embodiments, the PGPMs act as biocontrol agents of plant diseases by activating chemical and/or physical defenses of the host plants, i.e., activating induced systemic resistance (ISR) or systemic acquired resistance (SAR). In some embodiments, induction of resistance promoted by PGPMs of the present invention comprises active signaling in the salicylic acid pathway with induction of proteins related to pathogenesis (PR-proteins) or in the jasmonic acid and ethylene pathways. According to certain embodiments, when the PGPMs colonize the root system, constituents of the microorganism cell molecules act as biochemical signals, and the genes that encode for the synthesis of the PR-proteins are activated. In addition to PR-proteins, plants produce other enzymes of defense mechanisms, including peroxidases, phenylalanine ammonia-lyse (PAL), and polyphenoloxidase (PPO). Peroxidase and PPO are catalysts in the formation of lignin. PAL and other enzymes are involved in the formation of phytoalexins. In some embodiments, the PGPMs described herein induce plant resistance to diseases by increasing peroxidases, PPO and/or PAL production.

In some embodiments, the PGPMs of certain embodiments of the present invention promote the plant health, growth and/or yield via one or more of the mechanisms as described herein.

In some embodiments, the PGPMs of certain embodiments of the present invention are biofertilizers or biocontrol agents, which are compatible with organic farming.

Other aspects of the present embodiments contemplate isolated and/or cultured PGPMs. In certain aspects, the present invention provides isolated microbial strains (or PGPMs), isolated cultures thereof, biologically pure cultures thereof, and enriched cultures thereof. In some embodiments, the microbial isolate or culture comprises at least one microbial strain of the present invention as described herein. According to certain exemplary embodiments, the 16S rRNA gene of the microbial strain comprises a nucleotide sequence selected from SEQ ID NOs:1-11 and functional variants thereof. The microbial isolates or cultures promote the plant health, growth and/or yield, e.g., via one or more of the mechanisms as described herein in a plant cell of a plant or plant seed.

### Microbiological Compositions

The present invention provides microbial compositions that comprise at least one PGPM or microbial strain, such as a microbial strain selected from those described herein, or a culture thereof as described herein. According to certain embodiments, the composition further comprises a plant or plant seed. According to some embodiments, the plant or plant seed comprise at least one modified or transgenic trait. In some embodiments, the microbial composition comprises a microbial strain, wherein the 16S rRNA gene of said strain comprises a sequence selected from the group consisting of SEQ ID NOs.:1-11, a functional homolog or a culture thereof as described herein.

In some embodiments, the microbial composition comprises at least one microbial strain, wherein the 16S rRNA gene of the microbial strain comprises a nucleotide sequence that exhibits at least 85% sequence identity to SEQ ID NO:1; at least 85%, at least 90%, at least 91%, at least 92%, at least 93%, at least 94%, at least 95%, at least 96%, at least 97%, at least 98%, at least 99%, or at least 99.5% sequence identity to any one of the nucleotide sequences as set forth in SEQ ID NOs.:2-7; at least 97.5% sequence identity to SEQ ID NO:8: at least 94.5% sequence identity to SEQ ID NO:9; at least 97.5% identity to SEQ ID NO:10; at least 94.5 sequence identity to SEQ ID NO: 11, or a culture thereof.

According to certain embodiments, the composition comprises at least one Variovorax strain selected from the group consisting of strain S3167, strain S2492, and functional homologs thereof.

According to certain embodiments, strain S3167 or the functional homolog thereof is selected from the group consisting of:
a. a strain deposited under Accession Number NRRL No. B-67735;
b. a strain comprising 16S-rRNA sequence comprising a nucleic acid sequence at least 85% identical to SEQ ID NO:1; and/or at least 97.5% identical to SEQ ID NO:8; and/or at least 94.5% identical to SEQ ID NO:9; and
c. a strain comprising at least one genomic marker having at least 95% local identity to the nucleic acid sequence set forth in any one of SEQ ID NOs:29, 30, 31, 32, and 33 over 90% coverage.

According to certain embodiments, strain S2492 or the functional homolog thereof is selected from the group consisting of:
a. a strain deposited under Accession Number NRRL No. B-67736;
b. a strain comprising 16S-rRNA sequence comprising a nucleic acid sequence at least 85% identical to SEQ ID NO:1; and/or at least 97.5% identical to SEQ ID NO:10; and/or at least 94.5% identical to SEQ ID NO:11; and
c. a strain comprising a genomic nucleic acid marker having at least 95% local identity to a nucleic acid sequence set forth in any one of SEQ ID NOs:34, 35, 36, 37, and 38 over 90% coverage.

In some embodiments, the present invention provides a microbial composition comprising at least two, at least three, at least four, at least five, at least ten, or at least 20 microbial strains of the present invention or a culture thereof. According to certain embodiments, the composition further comprises a plant or a plant seed.

In some embodiments, the microbial composition comprises one or more Variovorax strain selected from the group consisting of S3167 (NRRL Deposit No. B-67735), S2492 (NRRL Deposit No. B-67736), and S2441, and at least one additional microbial strain selected from P0035_B2 (also referred to as S2145; NRRL Deposit No. B-67091), P0020_B1, P0047_A1 (also referred to as S2284; NRRL Deposit No. B-67102), P0033_E1 (also referred to as S2177), P0032_A8 (also referred to as S2181; NRRL Deposit No. B-67099), P0049_E7, P0042_A8 (also referred to as S2167), P0042_D5 (also referred to as S2165), P0042_B2 (also referred to as S2168; NRRL Deposit No. B-67096), P0042_B12 (also referred to as S2189), P0042_C2 (also referred to as S2173; NRRL Deposit No. B-67098), P0042_D10 (also referred to as S2172; NRRL Deposit No. B-67097), P0044_A3 (also referred to as S2476), P0018_A11, P0044_A5, P0047_E2, P0047_C1, P0038_D2 (also referred to as S2166), P0042_E1, P0047_E8, P0018_A1, P0058_B9 (also referred to as S2159; NRRL Deposit No. B-67092), P0054_E8 (also referred to as S2161; NRRL Deposit No. B-67094), P0054_F4 (also referred to as S2164), P0057_A3 (also referred to as S2160; NRRL Deposit No. B-67093), P0061_E11 (also referred to as S2142), P0019_A12 (also referred to as S2163; NRRL Deposit No. B-67095), P0147_D10 (also referred to as S2291; NRRL Deposit No. B-67104), P0147_G10 (also referred to as S2292; NRRL Deposit No. B-67105), P0160_F7 (also referred to as S2351), P0140_C10 (also referred to as S2300; NRRL Deposit No. B-67107), S2387, P0157_G5 (also referred to as S2303; NRRL Deposit No. B-67108, P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106), S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. B-67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRL Deposit No. B-67445), S2145-2 (NRRL Deposit No. B-67331), S2292-2 (NRRL Deposit No. B-67332), S2300-2 (NRRL Deposit No. B-67333), S2303-2 (NRRL Deposit No. B-67334), S2375-2 (NRRL Deposit No. B-67335), S2382-2 (NRRL Deposit No. B-67336), S2423-2 (NRRL Deposit No. B-67337), S2669-2 (NRRL Deposit No. B-67338), and any combination thereof, and strains derived therefrom, or cultures thereof.

According to certain embodiments, the composition further comprises a plant or plant seed. In some embodiments, the microbial composition comprises at least two, at least three, at least four, at least five, at least ten, or at least 20 or at least 30 and more microbial strains disclosed herein and a plant or plant seed. In another embodiment, the microbial composition comprises a plurality of strains disclosed herein.

In some embodiments, the microbial composition comprises at least one, at least two, at least three, at least four, at least five, at least ten, or at least 20 microbial strains selected from P0035_B2 (also referred to as S2145; NRRL Deposit No. B-67091), P0020_B1, P0047_A1 (also referred to as S2284; NRRL Deposit No. B-67102), P0033_E1 (also referred to as S2177), P0032_A8 (also referred to as S2181; NRRL Deposit No. B-67099), P0049_E7, P0042_A8 (also referred to as S2167), P0042_D5 (also referred to as S2165), P0042_B2 (also referred to as S2168; NRRL Deposit No. B-67096), P0042_B12 (also referred to as S2189), P0042_C2 (also referred to as S2173; NRRL Deposit No. B-67098), P0042_D10 (also referred to as S2172; NRRL Deposit No. B-67097), P0044_A3 (also referred to as S2476), P0018_A11, P0044_A5, P0047_E2, P0047_C1, P0038_D2 (also referred to as S2166), P0042_E1, P0047_E8, P0018_A1, P0058_B9 (also referred to as S2159; NRRL Deposit No. B-67092), P0054_E8 (also referred to as S2161; NRRL Deposit No. B-67094), P0054_F4 (also referred to as S2164), P0057_A3 (also referred to as S2160; NRRL Deposit No. B-67093), P0061_E11 (also referred to as S2142), P0019_A12 (also referred to as S2163; NRRL Deposit No. B-67095), P0147_D10 (also referred to as S2291; NRRL Deposit No. B-67104), P0147_G10 (also referred to as S2292; NRRL Deposit No. B-67105), P0160_F7 (also referred to as S2351), P0140_C10 (also referred to as S2300; NRRL Deposit No. B-67107), S2387, P0157_G5 (also referred to as S2303; NRRL Deposit No. B-67108, P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106), S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. B-67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRL Deposit No. B-67445), S2145-2 (NRRL Deposit No. B-67331), S2292-2 (NRRL Deposit No. B-67332), S2300-2 (NRRL Deposit No. B-67333), S2303-2 (NRRL Deposit No. B-67334), S2375-2 (NRRL Deposit No. B-67335), S2382-2 (NRRL Deposit No. B-67336), S2423-2 (NRRL Deposit No. B-67337), S2669-2 (NRRL Deposit No. B-67338), or strains derived therefrom, or cultures thereof.

According to certain embodiments, the composition further comprises a plant or plant seed. According to some embodiments, the plant or plant seed comprise at least one modified or transgenic trait. According to certain exemplary embodiments, the present invention provides a composition comprising one or more Variovorax microbial strains. In other embodiments the present invention provides a composition comprising one or more Variovorax paradoxus microbial strains.

According to certain embodiment, the present invention provides a composition comprising a synthetic microbial consortium. In some embodiments, a synthetic consortium comprises: (a) a first set of microbes comprising one or more microbes that promote plant health, growth, and/or yield; and (b) a second set of microbes comprising one or more microbes that increase (directly or indirectly) the competitive fitness of one or more of the microbes of the first set; wherein the first and the second sets of microbes are combined into a single mixture as a synthetic consortium. In one embodiment, the synthetic consortium comprises microbial strains not found together in nature. In another embodiment, the synthetic consortium comprises microbial strains not found in comparable concentrations relative to one another in nature. In some embodiments of a synthetic consortium, one or more microbes of the first set of microbes ((a) above) enhance nutrient availability and/or nutrient uptake of a plant. In some embodiments of a synthetic consortium, one or more microbes in the first set of microbes ((a) above) modulate plant hormone levels. In some embodiments of a synthetic consortium, one or more microbes in the first set of microbes ((a) above) demonstrate one or more of the activities selected from nitrogen fixation, IAA production, ACC deaminase activity, phosphate solubilization, and/or iron solubilization (and/or any other activities from which plant health, growth, and/or yield may be benefited). In some embodiments of a synthetic consortium, one or more microbes of the first set of microbes ((a) above) inhibit or suppress a plant pathogen (e.g., as a biological pesticide such as one selected from those described herein). In some embodiments of a synthetic consortium, one or more microbes in the second set of microbes ((b) above) directly increase the competitive fitness of one or more microbes in the first set of microbes ((a) above). In some embodiments, one or more microbes in the second set of microbes produce a metabolite that enhances the competitive fitness of one or more microbes in the first set of microbes. For example, one or more microbes in the second set of microbes produce a siderophore that enhances iron acquisition of one or more of the microbes in the first set of microbes. In some embodiments of a synthetic consortium, one or more microbes in the second set of microbes ((b) above) decrease the competitive fitness of a microorganism that is distinct from the microbes of the first or the second sets of microbes ((a) or (b) above), and potentially detrimental to (e.g., by inhibiting, competing with, excluding, or otherwise having a negative impact on) the fitness of one or more microbes in the first set of microbes ((a) above). In some embodiments of a synthetic consortium, one or more microbes in the second set of microbes ((b) above) produce a metabolite that is bactericidal, bacteriostatic or otherwise modulates growth of a microorganism that is distinct from the microbes of the first and the second sets of microbes, and that is detrimental to (e.g., by inhibiting, competing with, excluding, or otherwise having a negative impact on) the fitness of one or more microbes in the first set of microbes ((a) above). For example, one or more of the microbes in the second set of microbes ((b) above) produce a siderophore that inhibits the growth or fitness of a microorganism that is potentially detrimental to one or more microbes in the first set ((a) above). Thus, the function of the second set of microbes is to directly or indirectly increase the fitness or competitive fitness of the first set of microbes. In some embodiments of a synthetic consortium, the first and second set of microbes are combined and supplemented with an inert formulation component. In some embodiments, the synthetic consortium and compositions thereof promotes or enhances the health, growth and/or yield of a plant. In some embodiments, the synthetic consortium or a composition thereof according to the present invention is applied to a plant or plant seed.

In some embodiments, the microbial compositions described herein, such as any of the microbial compositions described above, further comprise an agriculturally effective amount of an additional substance, compound or composition, including, but not limited to, a nutrient, a fertilizer, an acaricide, a bactericide, a fungicide, an insecticide, a microbicide, a nematicide, a pesticide, or a combination thereof.

In some embodiments, the compositions are chemically inert; hence they are compatible with substantially any other constituents of the application schedule. The compositions may also be used in combination with plant growth affecting substances, such as fertilizers, plant growth regulators, and the like, provided that such compounds or substances are biologically compatible. The compositions may also be used in combination with biologically compatible pesticidal active agents as, for example, herbicides, nematicides, fungicides, insecticides, and the like.

In some embodiments, the microbial strains and compositions may furthermore be in the form of a mixture with at least one synergist compound. Synergists are compounds which increase the activity of the compositions without it being necessary for the synergist added to be active itself.

In some embodiments, the microbial strains and compositions may furthermore be in the form of a mixture with inhibitors (e.g., preservatives) which reduce the degradation of the active compositions after application in the habitat of the plant, on the surface of parts of plants or in plant tissues.

The active microbial strains and compositions may be used as a mixture with known fertilizers, acaricides, bactericides, fungicides, insecticides, microbicides, nematicides, pesticides, or any combinations thereof, for example in order to widen the spectrum of action or to prevent the development of resistances to pesticides in this way. In many cases, synergistic effects, i.e., the activity of the mixture can exceed the activity of the individual components. A mixture with other known active compounds, such as growth regulators, safeners and/or semiochemicals is also contemplated.

In some embodiments, the compositions may include at least one chemical or biological fertilizer. The amount of the at least one chemical or biological fertilizer employed in the compositions may vary depending on the final formulation as well as the size of the plant and/or seed to be treated. In some embodiments, the at least one chemical or biological fertilizer employed is about 0.1% w/w to about 80% w/w based on the entire formulation. In some embodiments, the at least one chemical or biological fertilizer is present in an amount of from about 1% w/w to about 60% w/w and in some embodiments from about 10% w/w to about 50% w/w.

The microbiological compositions optionally further include at least one biological fertilizer. Exemplary biological fertilizers that are suitable for use herein and can be included in a microbiological composition according to the embodiments of the present invention for promoting plant growth and/yield include microbes, animals, bacteria, fungi, genetic material, plant, and natural products of living organisms. In these compositions, the microorganism is isolated prior to formulation with an additional organism. For example, microbes selected from the group consisting of, but not limited to species of Achromobacter, Ampelomyces, Arthrobacter, Aureobasidium, Azospirillum, Azotobacter, Bacillus, Beauveria, Bradvrhizobium, Candida, Chaetorium, Cordyceps, Cryptococcus, Dabaryomyces, Delftia, Erwinia, Exophilia, Gliocladium, Herbaspirillum, Lactobacillus, Mariannaea, Microccocus, Paecilomyces, Paenibacillus, Pantoea, Pichia. Rhizobium, Saccharomyces, Sporobolomyces, Stenrotrophomonas, Ialaromyces, and Trichoderma can be provided in a composition with the microorganisms of the invention.

In some embodiments, the methods and compositions disclosed herein may include at least one chemical or biological pesticide, acaricide, bactericide, fungicide, insecticide, microbicide, nematicide, or any combination thereof. Each possibility represents a separate embodiment of the present invention. The amount of the at least one chemical or biological pesticide, acaricide, bactericide, fungicide, insecticide, microbicide, nematicide, or a combination thereof employed in the compositions can vary depending on the final formulation as well as the size of the plant and seed to be treated. In some embodiments, the at least one chemical or biological pesticide, acaricide, bactericide, fungicide, insecticide, microbicide, nematicide, or a combination thereof employed is about 0.1% w/w to about 80% w/w based on the entire formulation. In some embodiments, the at least one chemical or biological pesticide, acaricide, bactericide, fungicide, insecticide, microbicide, nematicide, or a combination thereof is present in an amount of from about 1% w/w to about 60% w/w and most preferably from about 10% w/w to about 50% w/w.

A variety of chemical pesticides may be used. Exemplary chemical pesticides include those in the carbamate, organophosphate, organochlorine, and pyrethroid classes. Also included are chemical control agents selected from the group consisting of, but not limited to, benomyl, borax, captafol, captan, chorothalonil, formulations containing copper; formulations containing dichlone, dicloran, iodine, zinc; fungicides selected from the group consisting of, but not limited to blastididin, cymoxanil, fenarimol, flusilazole, folpet, imazalil, ipordione, maneb, manocozeb, metalaxyl, oxycarboxin, myclobutanil, oxytetracycline, Pentachloronitrobenzene (PCNB), pentachlorophenol, prochloraz, propiconazole, quinomethionate, sodium aresenite, sodium DNOC, sodium hypochlorite, sodium phenylphenate, streptomycin, sulfur, tebuconazole, terbutrazole, thiabendazole, thiophanate-methyl, triadimefon, tricyclazole, triforine, validimycin, vinclozolin, zineb, and ziram. Each possibility represents a separate embodiment of the present invention.

In some embodiments, the methods and compositions disclosed herein include employing at least one biological pesticide. Exemplary biological pesticides that are suitable for use herein and can be included in a microbiological composition for preventing a plant pathogenic disease include microbes, animals, bacteria, fungi, genetic material, plant, and natural products of living organisms. In these compositions, the microorganism is isolated prior to formulation with an additional organism. For example, microbes including, but not limited to species of Anthrobacter, Ampelomyces, Aureobasidium, Bacillus, Beauveria, Candida, Chaetomium, Cordyceps, Crypiococcus, Dabaryomyces, Erwinia, Ficophilia, Gliocladium, Mariannaea, Paecilomyces, Paenibacillus, Pantoea, Pichia, Pseudomonas, Sporobolomyces, Streptomvces, Talaromyces, and Trichoderma can be provided in a composition with the microorganisms disclosed herein, with fungal strains of the Muscodor genus being exemplary embodiments. Each possibility represents a separate embodiment of the present invention.

Examples of fungi that may be combined in a composition with the microbial strains and compositions of the invention include, without limitation, Muscodor species, Aschersonia aleyrodis, Beauveria bassiana (“white muscarine”), Beauveria brongniartii, Chladosporium herbarum, Cordyceps clavulata, Cordyceps en tomorrhiza, Cordyceps facis, Cordyceps gracilis, Cordyceps melolanthae, Cordyceps militaris, Cordyceps myrmecophila, Cordyceps ravenelii, Cordyceps sinensis, Cordyceps sphecocephala, Cordyceps subsessilis, Cordyceps unilateralis, Cordyceps variabilis, Cordyceps washingtonensis, Culicinomyces clavosporus, Entomophaga grylli, Entomophaga maimaiga, Entomophaga muscae, Entomophaga praxibulli, Entomophthora plutellae, Fusarium lateritium, Glomus species, Hirsutella citriformis, Hirsutella thompsoni, Metarhizium anisopliae (“green muscarine”), Metarhizium flaviride, Muscodor albus, Neozygites floridana, Nomuraea rileyi, Paecilomyces farinosus, Paecilomyces fumosoroseus, Pandora neoaphidis, Tolypocladium cylindrosporum, Verticillium lecanii, Zoophthora radicans, and mycorrhizal species such as Laccaria bicolor. Each possibility represents a separate embodiment of the present invention. Other mycopesticidal species will be apparent to those skilled in the art.

In still further embodiments, the PGPM compositions, consortia and methods disclosed herein can be used to treat a genetically modified plant or seed or a transgenic planm or seed. As used herein, the term “genetically modified” is intended to mean any species containing a genetic trait, loci, or sequence that was not found in the species or strain or that was located in a different position or under different regulation in the genome of the species or strain prior to manipulation. A genetically modified plant may be transgenic, cis-genic, genome edited, or bred to contain a new genetic trait, loci, or sequence. A genetically modified plant may be prepared by means known to those skilled in the art, such as transformation by bombardment, by a Cas/CRISPR or TALENS system, or by breeding techniques. As used herein, a “trait” is a new or modified locus or sequence of a genetically modified plant, including, but not limited to, a transgenic plant. A trait may provide herbicide or insect resistance to the genetically modified plant. As used herein, a “transgenic” plant, plant part, or seed refers to a plant, plant part, or seed containing at least one exogenous gene that allows the expression of a polynucleotide or polypeptide not naturally found in the plant or not naturally located within the plant genome. The exogenous gene can thus be heterologous to the plant, or a plant endogenous gene not located in its natural position. The heterologous gene in a transgenic seed can originate, for example, from microorganisms of the species Bacillus, Rhizobium, Pseudomonas, Serratia. Trichoderma, Clavibacter, Glomus or Gliocladium.

Further embodiments of the present invention relate to a method of increasing the durability of plant pest compositions, comprising providing a plant protection composition to a plant or planted area, and providing the PGPM strains, cultures, compositions, and/or consortia described herein to the plant or planted area, wherein the PGPM strains, compositions, cultures and/or consortia have a different mode of action than the plant protection composition.

The present disclosure further provides methods and compositions that contain at least one of the isolated microbial strains or cultures thereof, such as any one of those described herein, and a carrier. The carrier may be any one or more of a number of carriers that confer a variety of properties, such as increased stability, wettability, dispersibility, etc. Wetting agents such as natural or synthetic surfactants, which can be nonionic or ionic surfactants or a combination thereof, can be included in a composition of the invention. Emulsions, such as water-in-oil emulsions can also be used to formulate a composition that includes at least one isolated microorganism of the present invention (see, for example, U.S. Pat. No. 7,485,451). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products (e.g., ground grain or beans, broth or flour derived from grain or beans), starch, sugar, or oil. The carrier may be an agricultural carrier. In certain exemplary embodiments, the carrier is a seed, and the composition may be applied or coated onto the seed or allowed to saturate the seed.

In some embodiments, the agricultural carrier may be soil or plant growth medium. Other agricultural carriers that may be used include fertilizers, plant-based oils, humectants, or combinations thereof. In some embodiments, an agricultural carrier does not include only water as a carrier. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, including, but not limited to, pesta (flour and kaolin clay), agar or flour-based pellets in loam, sand, or clay, and the like. Formulations may include food sources for the cultured organisms, such as barley, rice, or other biological materials such as seed, plant parts, sugar cane bagasse, hulls or stalks from grain processing, ground plant material (“yard waste”), compost, or wood from building site refuse, sawdust or small fibers from recycling of paper, fabric, or wood. Other suitable agricultural carriers are known to those skilled in the art.

In some embodiments, the carrier suitable for the compositions described herein is an organic carrier. The organic carriers include, but are not limited to, peat, turf, talc, lignite, kaolinite, pyrophyllite, zeolite, montmorillonite, alginate, press mud, sawdust, and vermiculite. Talc is a natural mineral referred as steatite or soapstone composed of various minerals in combination with chloride and carbonate. Chemically it is referred as magnesium silicate and available as powder form from industries suited for wide range of applications. Talc has relative hydrophobicity, low moisture equilibrium, chemical inertness, reduced moisture absorption and it prevents the formation of hydrate bridges which enable longer storage periods. Peat (turf) is a carbonized vegetable tissue formed in wet conditions by decomposition of various plants and mosses. Peat is formed by the slow decay of successive layers of aquatic and semi aquatic plants, such as sedges, reeds, rushes, and mosses. Press mud is a byproduct of sugar industries. Vermiculite is a light mica-like mineral used to improve aeration and moisture retention. In some embodiments, compositions with organic carriers as described herein are suitable for organic farming. Other suitable organic carriers are known to those skilled in the art.

The microbiological compositions that comprise isolated microbial strains or cultures thereof may be in a variety of forms, including, but not limited to, still cultures, whole cultures, stored stocks of cells, mycelium and/or hyphae (particularly glycerol stocks), agar strips, stored agar plugs in glycerol/water, freeze dried stocks, and dried stocks such as lyophilizate or mycelia dried onto filter paper or grain seeds. As defined herein, “isolated culture” or grammatical equivalents as used in this disclosure and in the art is understood to mean that the referred to culture is a culture fluid, pellet, scraping, dried sample, lyophilizate, or section (for example, hyphae or mycelia); or a support, container, or medium such as a plate, paper, filter, matrix, straw, pipette or pipette tip, fiber, needle, gel, swab, tube, vial, particle, etc. that contains a single type of organism. An isolated culture of a microbial antagonist is a culture fluid or a scraping, pellet, dried preparation, lyophilizate, or section of the microorganism, or a support, container, or medium that contains the microorganism, in the absence of other organisms.

In some embodiments, the compositions are in a liquid form. For example, in the liquid form, e.g., solutions or suspensions, the microorganisms of the present embodiments may be mixed or suspended in water or in aqueous solutions. Suitable liquid diluents or carriers include water, aqueous solutions, petroleum distillates, or other liquid carriers.

In some embodiments, the compositions are in a solid form. For example, solid compositions can be prepared by dispersing the microorganisms of the embodiments in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

In one embodiment, the microbial composition promotes plant health, growth and/or yield via one or more mechanisms by which PGPMs function, as described herein. In some embodiments, the compositions contemplated herein enhance the growth and yield of crop plants by acting as microbial fertilizers, biocontrol agents of plant diseases, and/or inducers of plant resistance. The compositions, similarly to other biofertilizer agents, may have a high margin of safety because they typically do not burn or injure the plant. In some embodiments, a biocontrol agent comprises a bacterium, a fungus, a yeast, a protozoan, a virus, an entomopathogenic nematode, a botanical extract, a protein, a nucleic acid, a secondary metabolite, and/or an inoculant.

As described herein, enhancing plant growth and plant yield may be affected by application of one or more of the compositions to a host plant or parts of the host plant. The compositions can be applied in an amount effective to enhance plant growth or yield relative to that in an untreated control. The active constituents are used in a concentration sufficient to enhance the growth of the target plant when applied to the plant. Effective concentrations may vary depending upon various factors such as, for example, (a) the type of the plant or agricultural commodity; (b) the physiological condition of the plant or agricultural commodity; (c) the concentration of pathogens affecting the plant or agricultural commodity; (d) the type of disease injury on the plant or agricultural commodity; (e) weather conditions (e.g., temperature, humidity); and (f) the stage of plant disease. Typical application concentrations are of about 10 to 1×10¹⁴ colony forming units (cfu) per seed, including about 1×10³ cfu/seed, or about 1×10⁴ cfu/seed, 1×10⁵ cfu/seed, or about 1×10⁶ cfu/seed, or about 1×10⁷ cfu/seed, or about 1×10⁸ cfu/seed, or about 1×10⁹ cfu/seed, or about 1×10¹⁰ cfu/seed, or about 1×10¹¹ cfu/seed, or about 1×10¹² cfu/seed, or about 1×10¹³ cfu/seed including about 1×10³ to 1×10⁸ cfu/seed about 1×10³ to 1×10⁷ cfu/seed, about 1×10³ to 1×10⁵ cfu/seed, about 1×10³ to 1×10⁶ cfu/seed, about 1×10³ to 1×10⁴ cfu/seed, about 1×10⁵ to 1×10⁹ cfu/seed, about 1×10³ to 1×10¹⁰ cfu/seed, about 1×10³ to 1×10¹¹ cfu/seed, about 1×10³ to 1×10¹² cfu/seed, about 1×10³ to 1×10¹³ cfu/seed, about 1×10⁴ to 1×10⁸ cfu/seed about 1×10⁴ to 1×10⁷ cfu/seed, about 1×10⁴ to 1×10⁵ cfu/seed, about 1×10⁴ to 1×10⁶ cfu/seed, about 1×10⁴ to 1×10⁹ & cfu/seed, about 1×10⁴ to 1×10¹⁰ cfu/seed, about 1×10¹¹, to 1×10⁹ cfu/seed, about 1×10⁴ to 1×10¹² cfu/seed about 1×10⁴ to 1×10¹³ cfu/seed, about 1×10⁵ to 1×10⁷ cfu/per seed, about 1×10⁵ to 1×10⁶ cfu/per seed, about 1×10⁵ to 1×10⁸ cfu/per seed, about 1×10⁵ to 1×10⁹ cfu/per seed, about 1×10⁵ to 1×10¹⁰ cfu/per seed, about 1×10⁵ to 1×10¹¹ cfu/per seed, about 1×10⁵ to 1×10¹² cfu/per seed, about 1×10⁵ to 1×10¹³ cfu/per seed, about 1×10⁶ to 1×10⁸ cfu/per seed, about 1×10⁶ to 1×10⁷ cfu/per seed, about 1×10⁶ to 1×10⁹ cfu/per seed, about 1×10⁶ to 1×10¹⁰ cfu/per seed, about 1×10⁶ to 1×10¹¹ cfu/per seed, about 1×10⁶ to 1×10¹² cfu/per seed, about 1×10⁶ to 1×10¹³ cfu/per seed, about 1×10⁷ to 1×10⁸ cfu/per seed, about 1×10⁷ to 1×10⁹ cfu/per seed, about 1×10⁷ to 1×10¹⁰ cfu/per seed, about 1×10⁷ to 1×10¹¹ cfu/per seed, about 1×10⁷ to 1×10¹² cfu/per seed, about 1×10⁷ to 1×10¹³ cfu/per seed, about 1×10⁸ to 1×10⁹ cfu/per seed, about 1×10⁸ to 1×10¹⁰ cfu/per seed, about 1×10⁸ to 1×10¹¹ cfu/per seed, about 1×10⁸ to 1×10¹² cfu/per seed, about 1×10⁸ to 1×10¹³ cfu/per seed, about 1×10⁹ to 1×10¹⁰ cfu/per seed, about 1×10⁹ to 1×10¹¹ cfu/per seed, about 1×10⁹ to 1×10¹² cfu/per seed, about 1×10⁹ to 1×10¹³ cfu/per seed, about 1×10¹⁰ to 1×10¹¹ cfu/per seed, about 1×10¹⁰ to 1×10¹² cfu/per seed, about 1×10¹¹ to 1×10¹³ cfu/per seed, about 1×10¹¹ to 1×10¹² cfu/per seed, about 1×10¹¹ to 1×10¹³ cfu/per seed, and about 1×10¹² to 1×10¹³ cfu/per seed. As used herein, the term “colony forming unit” or “cfu” is a unit capable of growing and producing a colony of a microbial strain in favorable conditions. The cfu count serves as an estimate of the number of viable structures or cells in a sample. In some embodiments, concentrations are those of from about 1 to about 100 mg dry bacterial mass per milliliter of carrier (liquid composition) or per gram of carrier (dry formulation). In some embodiments, the concentrations range from 1×10² to about 1×10¹⁰ cell/mL, such as the concentrations ranging from 1×10⁵ to 1×10⁹ cell/mL of the composition or carrier.

In some embodiments, the amount of one or more of the microorganisms in the compositions may vary depending on the final formulation as well as size or type of the plant or seed utilized. Preferably, the one or more microorganisms in the compositions are present in about 0.01% w/w to about 80% w/w of the entire formulation. In some embodiments, the dry weights of one or more microorganisms employed in the compositions is about 0.01%, 0.1%, 1%, 5% w/w to about 65% w/w and most preferably about 1% w/w to about 60% w/w by weight of the entire formulation.

The microbiological compositions may be applied to the target plant (or part(s) thereof) using a variety of conventional methods such as dusting, coating, injecting, rubbing, rolling, dipping, spraying, or brushing, or any other appropriate technique which does not significantly injure the target plant to be treated. Exemplary methods include, but are not limited to, the inoculation of growth medium or soil with suspensions of microbial cells and the coating of plant seeds with microbial cells and/or spores.

Also provided are methods of treating a plant by application of any of a variety of customary formulations in an effective amount to either the soil (i.e., in-furrow), a portion of the plant (i.e., drench) or on the seed before planting (i.e., seed coating or dressing). Customary formulations include solutions, emulsifiable concentrate, wettable powders, suspension concentrate, soluble powders, granules, suspension-emulsion concentrate, natural and synthetic materials impregnated with active compound, and very fine control release capsules in polymeric substances. In certain embodiments, the microbial compositions are formulated in powders that are available in either a ready-to-use formulation or are mixed together at the time of use. In either embodiment, the powder may be admixed with the soil prior to or at the time of planting. In an alternative embodiment, one or both of either the plant growth-promoting agent or biocontrol agent is a liquid formulation that is mixed together at the time of treating. One of ordinary skill in the art understands that an effective amount of the described compositions depends on the final formulation of the composition as well as the size of the plant or the size of the seed to be treated.

Depending on the final formulation and method of application, one or more suitable seed additives (additives) can also be introduced to the compositions. Adhesives such as carboxymethylcellulose and natural and synthetic polymers in the form of powders, granules or latexes, such as gum arabic, chitin, polyvinyl alcohol and polyvinyl acetate, as well as natural phospholipids, such as cephalins and lecithins, and synthetic phospholipids, trehalose, mannitol, sorbitol, myo-inositol, sophorose, maltotriose, glucose, (+)-galactose, methyl-beta-D-galactopyranoside, safener, a lipo-chitooligosaccharide, a triglucosamine lipoglycine salt, an isoflavone, and a ryanodine receptor modulator may be added to the present compositions.

In some embodiments, the compositions are formulated in a single, stable solution, or emulsion, or suspension. For solutions, the active chemical compounds are typically dissolved in solvents before the biological agent is added. Suitable liquid solvents include petroleum based aromatics, such as xylene, toluene or alkylnaphthalenes, aliphatic hydrocarbons, such as cyclohexane or paraffins, for example petroleum fractions, mineral and vegetable oils, alcohols, such as butanol or glycol as well as their ethers and esters, ketones, such as methyl ethyl ketone, methyl isobutyl ketone or cyclohexanone, strongly polar solvents, such as dimethylformamide and dimethyl sulphoxide. For emulsion or suspension, the liquid medium is water. In one embodiment, the chemical agent and biological agent are suspended in separate liquids and mixed at the time of application. In a preferred embodiment of suspension, the chemical agent and biological agent are combined in a ready-to-use formulation that exhibits a reasonably long shelf-life. In use, the liquid can be sprayed or can be applied foliarly as an atomized spray or in-furrow at the time of planting the crop. The liquid composition can be introduced in an effective amount on the seed (i.e., seed coating or dressing) or to the soil (i.e., in-furrow) before germination of the seed or directly to the soil in contact with the roots by utilizing a variety of techniques known in the art including, but not limited to, drip irrigation, sprinklers, soil injection or soil drenching. Optionally, stabilizers and buffers can be added, including alkaline and alkaline earth metal salts and organic acids, such as citric acid and ascorbic acid, inorganic acids, such as hydrochloric acid or sulfuric acid. Biocides can also be added and can include formaldehydes or formaldehyde-releasing agents and derivatives of benzoic acid, such as p-hydroxybenzoic acid.

### Seed Coating Formulations

According to certain aspects, the microbial strains, cultures and/or compositions described herein are formulated as a seed treatment on a plant seed. In some embodiments, plant seeds can be partially, or substantially uniformly coated with one or more layers of the microbial strains, cultures, and/or compositions disclosed herein using conventional methods, including but not limited to mixing, spraying or a combination thereof through the use of treatment application equipment that is specifically designed and manufactured to accurately, safely, and efficiently apply seed treatment products to seeds.

In some embodiments, plant seeds can be coated using a coating technology such as, but not limited to, rotary coaters, drum coaters, fluidized bed techniques, spouted beds, rotary mists or a combination thereof. Liquid seed treatments such as those of the present embodiments can be applied, for example, via either a spinning “atomizer” disk or a spray nozzle which evenly distributes the seed treatment onto the seed as it moves though the spray pattern. In certain embodiments, the seed is then mixed or tumbled for an additional period of time to achieve additional treatment distribution and drying. The seeds can be primed or unprimed before coating with the compositions to increase the uniformity of germination and emergence. In an alternative embodiment, a dry powder formulation can be metered onto the moving seed and allowed to mix until completely distributed.

According to other aspects, the present invention provides plant seeds treated with the subject microbial compositions, wherein the plant seed comprises at least one modified or transgenic trait. One embodiment provides the seeds having at least part of the surface area coated with a microbiological composition according to the present embodiments.

In certain embodiments, the microorganism-treated seeds have a microbial strain or spore concentration or microbial cell concentration from about 1×10² to about 1×10¹⁰ per seed. The seeds may also have more spores or microbial cells per seed. The microbial spores and/or cells can be coated freely onto the seeds or, preferably, they can be formulated in a liquid or solid composition before being coated onto the seeds. For example, a solid composition comprising the microorganisms can be prepared by mixing a solid carrier with a suspension of the spores until the solid carriers are impregnated with the spore or cell suspension. This mixture can then be dried to obtain the desired particles.

In some other embodiments, the microbial compositions contain functional agents capable of protecting the seeds from the harmful effects of selective herbicides such as activated carbon, nutrients (fertilizers), and other agents capable of improving the germination and quality of the products or a combination thereof.

Seed coating methods and compositions that are known in the art can be particularly useful when they are modified by the addition of one of the compositions disclosed herein. Such coating methods and apparatus for their application are disclosed in, for example, but not limited to, U.S. Pat. Nos. 5,918,413; 5,554,445; 5,389,399; 4,759,945; and 4,465,017. Seed coating compositions are disclosed, for example, in U.S. Patent Application Publication. No. US20100154299; U.S. Pat. Nos. 5,939,356; 5,876,739, 5,849,320; 5,791,084, 5,661,103; 5,580,544, 5,328,942; 4,735,015; 4,634,587; 4,372,080, 4,339,456; and 4,245,432.

A variety of additives can be added to the seed treatment formulations comprising the compositions disclosed herein. Binders can be added and include those composed preferably of an adhesive polymer that may be natural or synthetic without phytotoxic effect on the seed to be coated. The binder may be selected from polyvinyl acetates; polyvinyl acetate copolymers; ethylene vinyl acetate (EVA) copolymers; polyvinyl alcohols; polyvinyl alcohol copolymers; celluloses, including ethylcelluloses, methylcelluloses, hydroxymethylcelluloses, hydroxypropylcelluloses and carboxymethylcellulose; polyvinylpyrolidones; polysaccharides, including starch, modified starch, dextrins, maltodextrins, alginate and chitosans; fats; oils; proteins, including gelatin and zeins; gum arables; shellacs; vinylidene chloride and vinylidene chloride copolymers; calcium lignosulfonates; acrylic copolymers; polyvinylacrylates; polyethylene oxide; acrylamide polymers and copolymers; polyhydroxyethyl acrylate, methylacrylamide monomers; and polychloroprene.

Any of a variety of colorant additives may be employed, including organic chromophores classified as nitroso; nitro; azo, including monoazo, bisazo and polyazo; acridine, anthraquinone, azine, diphenylmethane, indamine, indophenol, methine, oxazine, phthalocyanine, thiazine, thiazole, triarylmethane, xanthene. Other additives that can be added include trace nutrients such as salts of iron, manganese, boron, copper, cobalt, nickel, molybdenum and zinc. A polymer or other dust control agent can be applied to retain the treatment on the seed surface.

In some exemplary embodiments, in addition to the microbial cells or spores, the coating can further comprise a layer of adherent. The adherent should be non-toxic, biodegradable, and adhesive. Examples of such materials include, but are not limited to, polyvinyl acetates; polyvinyl acetate copolymers; polyvinyl alcohols; polyvinyl alcohol copolymers; celluloses, such as methyl celluloses, hydroxymethyl celluloses, and hydroxymethyl propyl celluloses; dextrans; alginates; sugars; molasses; polyvinyl pyrrolidones; polysaccharides; proteins; fats; oils; gum arables; gelatins; syrups; and starches. More examples can be found in, for example, U.S. Pat. No. 7,213,367 and U.S. Patent Application Publication No. US20100189693.

Various additives, such as adherents, dispersants, surfactants, and nutrient and buffer ingredients, can also be included in the seed treatment formulation. Other seed treatment additives include, but are not limited to, coating agents, wetting agents, buffering agents, and polysaccharides. At least one agriculturally acceptable carrier may be added to the seed treatment formulation such as water, solids or dry powders. The dry powders can be derived from a variety of materials such as calcium carbonate, gypsum, vermiculite, talc, humus, activated charcoal, and various phosphorous compounds.

In some embodiments, the seed coating composition can comprise at least one filler which is an organic or inorganic, natural or synthetic component with which the active components are combined to facilitate its application onto the seed. In certain embodiments, the filler is an inert solid such as clays, natural or synthetic silicates, silica, resins, waxes, solid fertilizers (for example, ammonium salts), natural soil minerals, such as kaolins, clays, talc, lime, quartz, attapulgite, montmorillonite, bentonite or diatomaceous earths, or synthetic minerals, such as silica, alumina or silicates, in particular aluminum or magnesium silicates.

The seed treatment formulation may further include one or more of the following ingredients: other pesticides, including compounds that act only below the ground; fungicides, such as captan, thiram, metalaxyl, fludioxonil, oxadixyl, and isomers of each of those materials, and the like; herbicides, including compounds selected from glyphosate, carbamates, thiocarbamates, acetamides, triazines, dinitroanilines, glycerol ethers, pyridazinones, uracils, phenoxys, ureas, and benzoic acids; herbicidal safeners such as benzoxazine, benzhydryl derivatives, N,N-diallyl dichloroacetamide, various dihaloacyl, oxazolidinyl and thiazolidinyl compounds, ethanone, naphthalic anhydride compounds, and oxime derivatives; chemical fertilizers; biological fertilizers; and biocontrol agents such as other naturally-occurring or recombinant bacteria and fungi from the genera Rhizobium, Bacillus, Pseudomonas, Serratia, Trichoderma, Glomus, Gliocladium and mycorrhizal fungi. These ingredients may be added as a separate layer on the seed or alternatively, may be added as part of the seed coating composition of the embodiments.

In some embodiments, the amount of the composition or other ingredients used in the seed treatment should not inhibit germination of the seed or cause phytotoxic damage to the seed.

The formulation that is used to treat the seed in the compositions of the present invention may be in the form of a suspension; emulsion; slurry of particles in an aqueous medium (e.g., water); wettable powder; wettable granules (dry flowable); and dry granules. If formulated as a suspension or slurry, the concentration of the active ingredient in the formulation is about 0.5% to about 99% by weight (w/w), 5%-40% or as otherwise formulated by those skilled in the art.

In some embodiments, other conventional inactive or inert ingredients may be incorporated into the seed treatment formulation. Such inert ingredients include, but are not limited to, conventional sticking agents; dispersing agents such as methylcellulose, for example, serve as combined dispersant/sticking agents for use in seed treatments; polyvinyl alcohol; lecithin, polymeric dispersants (e.g., polyvinylpyrrolidone/vinyl acetate); thickeners (e.g., clay thickeners to improve viscosity and reduce settling of particle suspensions); emulsion stabilizers; surfactants; antifreeze compounds (e.g., urea), dyes, colorants, and the like. Further inert ingredients useful in the embodiments of this application can be found in McCutcheon's, vol. 1, “Emulsifiers and Detergents,” MC Publishing Company, Glen Rock, N.J., U.S.A., 1996. Additional inert ingredients useful in the embodiments of this application can be found in McCutcheon's, vol. 2, “Functional Materials,” MC Publishing Company, Glen Rock, N.J., U.S.A., 1996.

The coating formulations of the present invention may be applied to seeds by a variety of methods, including, but not limited to, mixing in a container (e.g., a bottle or bag), mechanical application, tumbling, spraying, and immersion. A variety of active or inert material can be used for contacting seeds with the microbial compositions, such as conventional film-coating materials including but not limited to water-based film coating materials such as SEPIRET™ (Seppic, Inc., N.J.) and OPACOAT™ (Berwind Pharm. Services, P.A.)

The amount of a composition according to the embodiments of the present invention that is used for the treatment of the seed will vary depending upon the type of seed and the type of active ingredients, but the treatment will comprise contacting the seeds with an agriculturally effective amount of the described composition. As discussed herein, an effective amount means that amount of the described composition that is sufficient to affect beneficial or desired results. An effective amount can be administered in one or more administrations.

In addition to the coating layer, the seed may be treated with one or more of the following ingredients: other pesticides including fungicides and herbicides; herbicidal safeners; fertilizers and/or biocontrol agents. These ingredients may be added as a separate layer or alternatively, may be added in the coating layer.

The seed coating formulations of the embodiments of the present invention may be applied to the seeds using a variety of techniques and machines, such as fluidized bed techniques, the roller mill method, rotostatic seed treaters, and drum coaters. Other methods, such as spouted beds may also be useful. The seeds may be pre-sized before coating. In some embodiments, after coating, the seeds are dried and then transferred to a sizing machine for sizing. Such procedures are known to a skilled artisan.

The microorganism-treated seeds may also be enveloped with a film overcoating to protect the coating. Such overcoatings are known in the art and may be applied using fluidized bed and drum film coating techniques, as well as any other suitable methods known in the art.

In another embodiment, microbial strains, isolates, cultures, and/or compositions of the present invention can be introduced onto a seed by use of solid matrix priming. For example, a quantity of a described composition can be mixed with a solid matrix material and then the seed can be placed into contact with the solid matrix material for a period to allow the composition to be introduced to the seed. The seed can then optionally be separated from the solid matrix material and stored or used, or the mixture of solid matrix material plus seed can be stored or planted directly. Solid matrix materials which are useful in may include polyacrylamide, starch, clay, silica, alumina, soil, sand, polyurea, polyacrylate, or any other material capable of absorbing or adsorbing the composition for a time and releasing that composition into or onto the seed. It is useful to make sure that the composition and the solid matrix material are compatible with each other. For example, the solid matrix material should be chosen so that it can release the composition at a reasonable rate, for example over a period of minutes, hours, days, or months.

In some embodiments, any plant seed capable of germinating to form a plant may be treated with the compositions contemplated herein. Suitable seeds include, but are not limited to, those of cereals, coffee, cole crops, fiber crops, flowers, fruits, legume, oil crops, trees, tuber crops, vegetables, as well as other plants of the monocotyledonous, and dicotyledonous species. In some embodiments, crop seeds are coated include, but are not limited to, bean, carrot, corn, cotton, grasses, lettuce, peanut, pepper, potato, rapeseed, rice, rye, sorghum, soybean, sugarbeet, sunflower, tobacco, and tomato seeds. In certain embodiments, barley or wheat (spring wheat or winter wheat) seeds are coated with the present compositions.

### Methods for Preparing the Composition

Cultures of the microorganisms may be prepared for use in the compositions of the present invention using techniques known in the art, including, but not limited to, standard static drying and liquid fermentation. Growth is commonly affected in a bioreactor. A bioreactor may be any appropriate shape or size for growing the microorganisms (PGPMs). A bioreactor may range in size and scale from 10 mL to liters to cubic meters and may be made of stainless steel or any other appropriate material as known and used in the art. The bioreactor may be a batch type bioreactor, a fed batch type or a continuous-type bioreactor (e.g., a continuous stirred reactor). For example, a bioreactor may be a chemostat as known and used in the art of microbiology for growing and harvesting microorganisms. A bioreactor may be obtained from any commercial supplier (See also Bioreactor System Design, Asenjo & Merchuk, CRC Press, 1995). For small scale operations, a batch bioreactor may be used, for example, to test and develop new processes, and for processes that cannot be converted to continuous operations.

Microorganisms or PGPMs grown in a bioreactor may be suspended or immobilized. Growth in the bioreactor is generally under aerobic conditions at suitable temperatures and pH for growth. Cell growth can be achieved at temperatures between 5 and 40° C., with the preferred temperature being in the range of 15 to 30° C., 15 to 28° C., 20 to 30° C., or 15 to 25° C. The pH of the nutrient medium can vary between 4.0 and 9.0, but the preferred operating range is usually slightly acidic to neutral at pH 4.0 to 7.0, or 4.5 to 6.5, or pH 5.0 to 6.0. Typically, maximal cell yield is obtained in 18-96 hours after inoculation.

Optimal conditions for the cultivation of the microorganisms of the present invention may depend upon the particular strain. However, by virtue of the conditions applied in the selection process and general requirements of most microorganisms, a person of ordinary skill in the art would be able to determine essential nutrients and conditions. The microorganisms or PGPMs would typically be grown in aerobic liquid cultures on media which contain sources of carbon, nitrogen, and inorganic salts that can be assimilated by the microorganism and supportive of efficient cell growth. Exemplary (but not limiting) carbon sources are hexoses such as glucose, but other sources that are readily assimilated such as amino acids, may be substituted. Many inorganic and proteinaceous materials may be used as nitrogen sources in the growth process. Exemplary (but not limiting) nitrogen sources are amino acids and urea but others include gaseous ammonia, inorganic salts of nitrate and ammonium, vitamins, purines, pyrimidines, yeast extract, beef extract, proteose peptone, soybean meal, hydrolysates of casein, distiller's solubles, and the like. Among the inorganic minerals that can be incorporated into the nutrient medium are the customary salts capable of yielding calcium, zinc, iron, manganese, magnesium, copper, cobalt, potassium, sodium, molybdate, phosphate, sulfate, chloride, borate, and like ions. In some embodiments, potato dextrose liquid medium for fungal strains and R2A broth premix for bacterial strains is used.

Methods for Using the Microbial Strains, Cultures, and/or Compositions

Other aspects provide a method for treating a plant or a plant seed, comprising a step of exposing or contacting a plant or plant seed with a microbial strain, isolate, culture, and/or composition as described herein.

Other aspects provide a method for enhancing the growth or yield of a plant, said method comprising applying an effective amount of a microbial strain, isolate, culture, and/or composition as described herein to the plant, part thereof or to the plant's surroundings. Another aspect provides a method for preventing, inhibiting or treating the development of a pathogenic disease of a plant, said method comprising applying an effective amount of a microbial strain, isolate, culture and/or composition as described herein to the plant, part thereof or to the plant's surroundings. In some embodiments of the methods, the microbial strain is grown in a growth medium or soil of a host plant prior to or concurrent with the host plant growth in said growth medium or soil. In some embodiments, the microbial strain is established as an endophyte on said plant. In some embodiments of the above method, a microbial strain (PGPM) is applied to the plant (or a part thereof) or to the plant's surroundings (e.g., immediate soil layer or rhizosphere) in a culture or a composition at a concentration that is at least 2×, 5×, 10×, 100×, 500×, or 1000× the concentration of the same microbial strain found in nature or detected in an untreated control plant (or a part thereof) or the control plant's surroundings, respectively. In some embodiments, upon or after application, the concentration of the microbial strain (PGPM) in the treated plant (or a part thereof) or the plant's surroundings (e.g., immediate soil layer or rhizosphere) is at least 2×, 5×, 10×, 100×, 500×, or 1000× the concentration of the same microbial strain found or detected in an untreated control plant (or a part thereof) or the control plant's surroundings. In some embodiments of the above method, a microbial strain (PGPM) is applied to the plant (or a part thereof) or to the plant's surroundings (e.g., immediate soil layer or rhizosphere) in a culture or a composition at a concentration of at least 1×10² CFU/mL. In some embodiments, concentration ranges from about 1×10² to about 1×10¹⁰ CFU/mL, such as the concentrations ranging from 1×10⁵ to 1×10⁹ CFU/mL. In some embodiments, application of a microbial strain (PGPM) to the plant (or a part thereof) or to the plant's surroundings (e.g., immediate soil layer or rhizosphere) in a culture or a composition at a concentration that is at least 1×10⁶ CFU/mL leads to a concentration of the microbial strain in the treated plant, plant part or the plant's surroundings that is at least 2× the amount of the strain found in the untreated plant or its surroundings.

In some embodiments of the above method, the microbial strain is established as an endophyte on the plant and the seed offspring of the plant after application. In some embodiments of this aspect, the microbial endophyte introduced into the plant may be an endophytic microorganism having a plant growth-promoting activity, a biological control activity, or a combination of both activities. A variety of methods previously found effective for the introduction of a microbial endophyte into cereal grass species are known in the art. Examples of such methods include those described in U.S. Pat. Appl. No. 20030195117A1, U.S. Pat. Appl. No. 20010032343A1, and U.S. Pat. No. 7,084,331. In some embodiments, the microbial strain, isolate, culture, and/or composition is applied to one or more places selected from the soil, a seed, a root, a flower, a leaf, a fruit, a portion of the plant or the whole plant. In this aspect, the microbial strain, culture or composition may be delivered to the plant by any of the delivery system described herein.

Examples of phytopathogenic diseases that are suitable for applications of the methods and materials include, but are not limited to, diseases caused by a broad range of pathogenic fungi. The methods of the present embodiments are preferably applied against pathogenic fungi that are important or interesting for agriculture, horticulture, plant biomass for the production of biofuel molecules and other chemicals, and/or forestry. In some embodiments, the pathogenic fungi are pathogenic Pseudomonas species (e.g., Pseudomonas solanacearum), Xylella fastidiosa; Ralstonia solanacearum, Xanthomonas campestris, Erwinia amylovora, Fusarium species, Phytophthora species (e.g., P. infestans), Botrytis species, Leptosphaeria species, powdery mildews (Ascomycota) and rusts (Basidiomycota), etc.

Non-limiting examples of plant pathogens of interest include, for instance, Acremontum strictum, Agrobacterium tumefaciens, Alternaria alternata, Alternaria solani, Aphanomyces euteiches, Aspergillus fimigatus, Athelia rolfsii, Aureobasidium pullulans, Bipolaris zeicola, Botrytis cinerea, Calonectria kyotensis, Cephalosporium maydis, Cercospora medicaginis, Cercospora sojina, Colletoirichum coccodes, Colletotrichum fragariae, Colletotrichum graminicola, Coniella diplodiella, Coprinopsis psychromorbida, Corynespora cassiicola, Curvularia pallescens, Cylindrocladium crotalariae, Diplocarpon earliamim, Diplodia gossvina, Diplodia spp., Epicoccum nigrum, Eysiphe dehor acearum, Fusarium graminearum, Fusarium oxysporum. Fusarium oxvsporum f.sp. tuberosi, Fusarium proihferatum var. proiferatum, Fusarium solani, Fusarium verticillioides, Ganoderma boninense, Geotrichum candidum, Glomerella tucumanensis, Guignardia bidwellii, Kabatiella zeae, Leptosphaerulina briosiana, Leptotrochila medicaginis, Macrophomina, Macrophomina phaseolina, Magnaporthe grisea, Magnaporthe oryzae, Microsphaera manshurica, Monilinia fructicola, Mycosphaerella fijiensis, Mycosphaerella fragariae, Nigrospora oryzae, Ophiostoma ulmi, Pectobacterium carotovorum, Pellicularia sasakii (Rhizoctonia solani), Peronospora manshurica, Phakopsora pachyrhizi, Phoma foveata, Phoma medicaginis, Phomopsis longicolla, Phytophthora cinnamomi, Phytophthora erythroseptica, Phytophthora fragariae, Phytophthora infestans, Phytophthora medicaginis, Phytophthora megasperma, Phytophthora palmivora, Podosphaera leucotricha, Pseudopeziza medicaginis, Puccinia graminis subsp. Tritici (UG99), Puccinia sorghi, Pyricularia grisea, Pyricularia oryzae, Pythium ultimum, Pythium aphanidermatum, Rhizoctonia solani, Rhizoctonia zeae, Rosellinia sp., Sclerotinia sclerotiorum, Sclerotinina trifoliorum, Sclerotium rolfsii, Septoria glycines, Septoria lycopersici, Setomelanomma turcica, Sphaerotheca macularis, Spongospora subterranea, Stemphylium sp, Synchytrium endobioticum, Thecaphora (Angiosorus), Thielaviopsis, Tilletia indica, Trichoderma viride, Ustilago maydis, Verticillium albo-atrum, Verticillium dahliae, Verticillium dahliae, Xanthomonas axonopodis, or Xanthomonas oryzae pv. oryae.

In some embodiments, the methods and materials are useful in suppressing the development of the pathogens Aspergillus fumigatus, Botrytis cinerea, Cerpospora betae, Colletotrichum sp., Curvularia spp., Fusarium sp., Ganoderma boninense, Geotrichum candidum, Gibberella sp., Monographella sp., Mycosphaerella fijiensis, Phytophthora palmivora, Phytophthora ramorum, Penicillium sp., Pythium ultimum, Pythium aphanidermatum, Rhizoctonia solani, Rhizopus spp., Schizophyllum spp., Sclerotinia sclerotiorum, Stagnospora sp., Verticillium dahliae, or Xanthomonas aronopodis. In some embodiments, the methods and materials may be used to suppress the development of several plant pathogens of commercial importance, including Fusarium graminearum NRRL-5883, Monographella nivalis ATCC MYA-3968, Gibberella zeae ATCC-16106, Stagnospora nodurum ATCC-26369, Colletotrichum graminicola ATCC-34167, and Penicillium sp. pathogens.

In some embodiments, the method for enhancing the growth or yield of a plant, including any of such methods described herein, further comprises a step of processing soil before planting a plant, a plant seed or a plant seedling in said soil. In some embodiments, the soil is fully or partially sterilized in the soil processing step. In some embodiments, the soil processing method comprises making a microwave radiator move into soil, and thereafter radiating microwaves from the microwave radiator to soil to be processed. Examples of such a method can be found, e.g., in US 20060283364. In some embodiments, the soil is fully or partially sterilized by autoclaving (e.g., at 121° C., 1 h or other similar conditions) or by gamma (γ)-irradiation (50 kGy). In some embodiments, the soil is fully or partially sterilized by heating, steaming or gassing with ethylene oxide. In some embodiments, the soil is partially or fully sterilized by soil solarization. Soil solarization is an environmentally friendly method of using solar power for soil processing (e.g., sterilization) by mulching the soil and covering it with tarp, usually with a plastic (e.g. transparent polyethylene) cover, to trap solar energy. Other suitable soil processing methods are known to those skilled in the art.

In some embodiments, the method for enhancing the growth or yield of a plant comprises (a) processing the soil before planting the plant, plant seed or seedling thereof in said soil; (b) planting the plant, plant seed or seedling thereof in the soil processed in step (a); and (3) applying an effective amount of a microbial strain, isolate, culture, and/or composition as described herein to the plant, plant seed or seedling, or surroundings thereof. In some embodiments, the soil is fully sterilized. In some embodiments, the soil is partially sterilized. In some embodiments, the soil is processed by autoclaving in step (a).

### Delivery Systems

Microbial stains, isolates or cultures thereof, or microbial compositions may be delivered through several means. In some embodiments, they are delivered by seed treatment, seed priming, seedling dip, soil application, foliar spray, fruit spray, hive insert, sucker treatment, sett treatment, and a multiple delivery system.

In some embodiments, the microbial strains, cultures thereof or compositions comprising the same, as described herein, may be delivered by direct exposure or contact with a plant, or a plant seed. In some embodiments, the seed can be coated with a microbial strain (or an isolate or a culture thereof) or a composition thereof. Seed treatment with PGPMs may be effective against several plant diseases.

In some embodiments, the microbial strains, isolates, cultures or compositions, as described herein, can be delivered by direct exposure or contact with a plant seed during seed priming process. Priming with PGPMs may increase germination and improve seedling establishment. Such priming procedures may initiate the physiological process of germination but prevents the emergence of plumule and radicle. It has been recognized that initiation of the physiological process helps in the establishment and proliferation of the PGPMs on the spermosphere.

In some embodiments, the microbial strains, isolates, cultures thereof or compositions comprising the same, as described herein, can be delivered by seedling dip. Plant pathogens often enter host plants through root. In some embodiments, protection of rhizosphere region by prior colonization with PGPMs prevents the establishment of a host-parasite relationship.

In some embodiments, the microbial strains, isolates, cultures or compositions, as described herein, can be delivered by direct application to soil. Soil is the repertoire of both beneficial and pathogenic microbes. In some embodiments, delivering PGPMs to soil can suppress the establishment of pathogenic microbes.

In some embodiments, the microbial strains, isolates, cultures or compositions, as described herein, can be delivered by foliar spray or fruit spray. In some embodiments, delivering PGPMs directly to plant foliage or fruit can suppress pathogenic microbes contributing to various foliar diseases or post-harvest diseases.

In some embodiments, the microbial strains, isolates, cultures or compositions are delivered by hive insert. Honey bees and bumble bees serve as a vector for the dispersal of biocontrol agents of diseases of flowering and fruit crops. In some embodiments, a dispenser can be attached to the hive and loaded with the PGPMs, optionally in combination with other desired agents.

In some embodiments, the microbial strains, isolates, cultures or compositions are delivered by sucker treatment or sett treatment. PGPMs can plant a vital role in the management of soil-borne diseases of vegetatively propagated crops. The delivery of PGPMs varies depending upon the crop. For crops such as banana, PGPMs may be delivered through sucker treatment (e.g., sucker dipping). For crops such as sugarcane, PGPMs may be delivered through sett treatment (e.g., sett dipping).

In some embodiments, the microbial strains, isolates, cultures or compositions are delivered by a multiple delivery system comprising two or more of the delivery systems as described herein.

Plant Varieties and Seed Offspring Infected with a Microbial Strain

Also provided, in other aspects of the present invention is an artificially infected plant created by artificially introducing a microbial strain disclosed herein to the plant. In some embodiments of this aspect, the microbial strain introduced to the plant may be an endophytic microorganism having a plant growth-promoting activity, a biological control activity, or a combination of both activities. In some embodiments, the microbial strain is established as an endophyte in the plant or a progeny thereof (e.g., the seed offspring) that is exposed to or treated with a microbial (endophytic) strain, isolate, culture or composition thereof as described herein. Accordingly, another embodiment provides a seed of the artificially infected plant, comprising the microbial endophyte disclosed herein.

A variety of methods previously found effective for the introduction of a microbial endophyte into, e.g. cereal grass species are known in the art. Examples of such methods include those described in U.S. Patent Application Publication No. 20030195117A1, U.S. Patent Application Publication No. 20010032343A1, and U.S. Pat. No. 7,084,331, among others.

In some embodiments, after artificial infection, a DNA sequence of the isolated endophytic microorganism is amplified by PCR and the endophyte is confirmed by carrying out a homology search for the DNA sequence amplified. In some embodiments, a foreign gene that expresses an identifiable means is introduced into the above-mentioned endophytic microorganism, and the presence of the colonization of the above-mentioned endophytic microorganism infecting the plant is confirmed by the above-identifiable means using the foreign gene.

### Suitable Plants

In principle, the methods and compositions of the present invention may be deployed for any plant species. Monocotyledonous as well as dicotyledonous plant species are particularly suitable. The methods and compositions are preferably used with plants that are important or interesting for agriculture, horticulture, for the production of biomass used in producing liquid fuel molecules and other chemicals, and/or forestry.

In still another embodiment, the PGPM compositions, consortia and methods disclosed herein can be used to treat transgenic seeds. A transgenic seed refers to the seed of plants containing at least one exogenous gene that allows the expression of a polypeptide or protein not naturally found in the plant. The exogenous gene in a transgenic seed can ne heterologous gene originated, for example, from microorganisms of the species Bacillus, Rhizobium, Pseudomonas. Serratia, Trichoderma, Clavibacter, Glomus or Gliocladium.

Thus, embodiments of the present invention have use over a broad range of plants, preferably higher plants pertaining to the classes of Angiospermae and Gymnospermae. Plants of the subclasses of the Dicotylodenae and the Monocotyledonae are particularly suitable. Dicotyledonous plants belong to the orders Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Mvricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, ScroAphlariales, Theales, Trochodendrales, Urmbellales, Urticales, and Violates, Monocotyledonous plants belong to the orders Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, iriuridales, Typhales, and Zingiberales. Plants belonging to the class of the Gymnospermae are Cycadales, Ginkgoales, Gnetales, and Pinales.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Fphedra, Frianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Thnacetum, Taxus, Theobroma, Triticosecale, Triticum, Uniola, Veratrum, Vinca, Vitis, and Zea.

The methods and compositions may be used in plants that are important or interesting for agriculture, horticulture, biomass for the production of biofuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza saliva (rice), Helianthus annuus (sunflower), Medicago saliva (alfalfa), Beta vulgaris (sugarbeet), Pennisetum glaucum (pearl millet), Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus), Triticosecale spp. (triticum—wheat X rye), Bambuseae (Bamboo), Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzofiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Cofea arabica (coffee), Vitis vinifera (grape), Ananas comosus (pineapple), Capsicum amnum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solamum melongena (eggplant), Papaver somniferum (opium poppy), Papaver orientale, Taxus baccata, Taxus brevifolia, Artemisia annua, Cannabis sativa, Camptotheca acuminate, Catharanthus roseus, Tinca rosea, Cinchona officinalis, Coichicum autumnale, Veratrum californica, Digitalis lanata, Digitalis purpurea, Dioscorea spp., Andrographs paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Agrostis spp. (bentgrass), Populus tremuloides (aspen), Pimus spp. (pine), Abies spp. (fir), Acer spp. (maple), Hordeum vulgare (barley), Poa pratensis (bluegrass), Lolium spp. (ryegrass), Phleum pratense (timothy), and conifers. Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum—wheat X rye), and Bambuseae (Bamboo); and starch-based energy crops like Zea mays (corn) and Manihot esculenta (cassava); and sugar-based energy crops like Saccharum sp. (sugarcane), Beta vulgaris (sugarbeet), and Sorghum bicolor (L.) Moench (sweet sorghum); and biofuel-producing energy crops like Glycine max (soybean), Brassica napus (canola), Helianthus annuus (sunflower), Carthamus tinctorius (safflower), Jatropha curcas (Jatropha), Ricinus communis (castor), Elaeis guineensis (African oil palm), Elaeis oleifera (American oil palm), Cocos nucifera (coconut), Camelina sativa (wild flax), Pongamia pinnata (Pongam), Olea europaea (olive), Linum usitatissimum (flax), Crambe abyssinica (Abyssinian-kale), and Brassica juncea.

In some embodiments, the methods and compositions may be used in corn, including but not limited to, flour corn (Zea mays var. amylacea), popcorn (Zea mays var. everta), dent corn (Zea mays var. indentata), flint corn (Zea mays var. indurate), sweet corn (Zea mays var. saccharata and Zea mays var. rugosa), waxy corn (Zea mays var. ceratina), amylomaize (Zea mays), pod corn (Zea mays var. tunicata Larranaga ex A. St. Hil.), and striped maize (Zea mays var. japonica). In some embodiments, the methods and compositions are used in sweetcorn.

This disclosure will be better understood from the Examples which follow. However, one skilled in the art will readily appreciate that the specific methods and results discussed are merely illustrative of the disclosure as described more fully in the embodiments.

### EXAMPLES

### Example 1: Collection of Soil Samples and Sequencing of Soil Microorganisms

Soil samples were collected from agricultural fields. For instance, soil samples were collected from corn fields in the United States and Australia. From each field corn plants at V4-V10 stage were selected, removed from the ground, and soil was collected. For each plant, height and weight were recorded, soil attached to the roots was collected for cultivation and DNA extraction, and bulk soil surrounding the root structure was collected for soil chemistry analysis and archiving. Soil samples not associated with plants were also collected from multiple locations in the field for baseline soil chemistry analysis and DNA extractions. The present invention contemplates plant growth-promoting microbes (PGPMs) identified and isolated from any suitable types of environmental materials, such as samples collected from, without limitation, soil, rock, plants, animals, organic debris, water, aerosols, etc. The present invention also contemplates PGPMs isolated from cultures inoculated with environmental materials and containing a carbon source, essential nutrients (including vitamins, trace metals and a source of calcium, phosphorus, sulfur and nitrogen), and optionally including a buffer to maintain pH.

Root associated soil samples (about 0.5 g) were collected in triplicate from the rhizosphere of corn plants for DNA extraction and sequencing. Samples were placed into 2-mL screw-cap centrifuge tubes containing a sterile ceramic bead matrix consisting of one 4-mm glass bead (GSM-40), 1.0 g of 1.4- to 1.6-mm zirconium silicate beads (SLZ-15) and 0.75 g of 0.070- to 0.125-mm zirconium silicate beads (BSLZ-1) obtained from Cero Glass (Columbia, Tenn.). Samples were kept cool and transported to the laboratory for DNA extraction.

Samples were mechanically lysed using a FastPrep FP 120 instrument (Bio-101, Vista, Calif.) at 6.5 m/sec for 45 sec in 1 ml phosphate buffer (200 mM sodium phosphate, 200 mM NaCl, 20 mM EDTA, pH 8.0) and 10% SDS (sodium dodecyl sulfate). Lysed samples were centrifuged at 13,000×g for 5 min at 4° C. to separate the supernatant with DNA and particulate matter. Supernatants were transferred into new 1.5-mL centrifuge tubes and further purified by adding 500 μl phenol-chloroform-isoamyl alcohol (25:24:1) and centrifuging at 13,200×g for 5 min at room temperature. The separated aqueous phase containing the DNA was collected for final purification on QIAprep Plasmid Spin columns (Qiagen, Valencia, Calif.) following manufacturer's instructions.

Identification of key organisms was performed by first extracting genomic DNA and then using 16S rRNA next generation sequencing (NGS) to generate environmental microbial profiles from agricultural fields following the methods of Patin et al. (Microb. Ecol. 65:709-719, 2013). Correlation analysis of microbe 16S sequence tags and desired target phenotypes, included but not limited to, grain yield, plant biomass, plant height, drought tolerance score, and anthesis to silking interval, determined the organisms of interest.

### Example 2: Identification of Microbial Consortia

The corn plants for sampling were at the V3-V10 stage of development and were chosen based upon being either under or over-performing plants based on visual inspection and comparison with neighboring plants. Under-performing plants were chosen based upon being equal or smaller in size to neighboring plants which collectively presented as smaller in size with the average size of plants across the entire field. Over-performing plants were chosen based upon being greater in size than the average size of plants across the general area or entire field. Another criterion for choosing an over-performing plant was that its immediate neighbors were also over-performing relative to the size of plants in the general area or entire field. Plants were collected in pairs that each included an under- and over-performing plant that were located within 5 meters of one another. Between 6-18 pairs of plants were collected from each field.

Prior to sampling, the height of each plant was determined by extending the upper leaves vertically to the highest point and measuring this level. The weight of the plant was determined post-sampling by removing the entire above soil portion of the plant and transferring into a sealed Ziploc quart size bag. The sealed bags were used to minimize variability due to water evaporation from the plant post-harvest. The weight of the plant was determined within approximately 1 hour after collection.

Corn root-associated soil sampling was conducted by digging up the corn plants with a shovel and carefully excavating roots with a sterile stainless-steel spatula. Soil clinging to the roots was removed directly into 2 ml centrifuge tubes containing beads for cell lysis and DNA extraction and profiling were performed as described in Example 1 (see Patin et al. Microb. Ecol. 65:709-719, 2013).

To compare microbial communities associated with corn roots from plants from different fields, the heights and weights of each plant collected from the same field were normalized. Several different normalization methods were deployed that included Z-scores, interpolation of the values between 0-1 and percent rank. The reason for normalizing the values was to enable comparison of plants between fields that, in some cases, were of different sizes because of different plant genetics, planting dates, soil types, weather, etc.

Approximately 100,000 or more V5V6 16S rRNA sequence tags were generated from each sample. Pearson correlation values were determined for the percent abundance of each 16S rRNA sequence tag and the normalized corn plant weight, height, yield or other parameters of interest across more than 300 microbial profiles from fields in Brentwood and Woodland, Calif., Wells, Minn., and Queensland, Australia. Bacterial 16S rRNA sequence tags with the highest correlation to several parameters of interest were identified. The 16S rRNA sequence tags with the highest correlation to plant performance (normalized plant height or weight, grain yield and drought tolerance) were of primary importance.

Cultivation screens were also performed from the same samples where the root-associated microbial communities were resolved by 16S rRNA gene profiling. More than 50,000 isolates were recovered by cultivating on seven different solid medium formulations. The identity of the isolates was determined by PCR-amplifying a portion of the 16S rRNA gene comprising the V1-V9 variable regions. The sequences were trimmed to the same V5V6 region as used for the 16S rRNA gene profiles conducted above. This step allowed for cross indexing between the cultivation and 16S rRNA gene profiling data.

Strains identified by 16S region and the 16S rRNA sequence ID Nos. are presented in Table 1 hereinbelow.

Additionally, plant growth promoting microbes (PGPM) are known to form cooperative functional groups, or consortia. Some members of a growth promoting consortium may not influence the plant directly, but instead influence the other microbes of the group. By distinguishing which sequence tags consistently correlate to other tags associated with high yield, across several geographic locations and years, common members of plant-associated consortia may be more easily recognized. Specific microbial strains that contribute to high yield may be different across all fields, but the strains that become supporting members in a plant associated consortia may be more consistent across sites. Herein, we give one such example of two microbe strains, comprising one 16S V5V6 sequence tag, that frequently co-occur with yield-associated microbes across spatial and temporal variables. These microbes may be acting as keystone species, and have the potential to increase the survival and functioning of native and non-native PGPM species.

In field trials, Niastella gongjuensis (S2876, NRRL No. B-67448) was found to increase yield potential. Niastella gongjuensis (S2876, NRRL No. B-67448) was selected as a field-testing candidate partly because the 16S rRNA tag it contained directly correlated to yield at Brentwood, Calif., in 2014. Additional 16S rRNA tags from the Brentwood data set were then identified for their potential to share functional interactions with the primary plant performance-correlated microbe of interest, Niastella gongjuensis (S2876, NRRL No. B-67448). To identify potential consortium members, distribution of the 16S rRNA sequence tags best correlated to plant performance were compared with every other sequence tag in the data set to identify co-distributing sequences. A ranked list of Pearson correlations of these comparisons was created to reveal candidate consortium members for each primary plant performance-correlated sequence tag. The sequence tags of candidate consortium members may or may not also have their own correlations to plant performance metrics within the same or other data sets. Consortium candidates that did independently correlate to plant performance metrics were of greater interest than those that did not. 16S v5v6 rRNA sequence tags identified include SEQ ID NOs:1-7.

Cultivated strains corresponding to the identified 16S rRNA sequence tags of interest were recovered and advanced to test their ability to improve plant performance, including strain S3167 Variovorax paradoxus (NRRL No. B-67735), strain S2492 Variovorax paradoxus (NRRL No. B-67736), and strain S2441 Variovorax ginsengisoli. 16S rRNA sequence tags for the selected strains include SEQ ID NOs:8-20.

TABLE 1
Identified strains

| SEQ ID | Strains Identified by | 16S | Species |
|---|---|---|---|
| NO: | 16S Region | Region | Name |
| 1 | S3167 (NRRL No. B-67735) and | v5v6 | Variovorax |
| | S2492 (NRRL No. B-67736) | | paradoxus |
| 2 | S2441 | v5v6 | Variovorax |
| | | | ginsengisoli |
| 3 | S2876 (NRRL No. B-67448) | v5v6 | Niastella |
| | | | gongjuensis |
| 4 | S2550 | v5v6 | Streptomyces |
| | | | rishiriensis |
| 5 | TXv5v6-0061239 | v5v6 | Ferruginibacter |
| | | | lapsinanis |
| 6 | TXv5v6-2170581 | v5v6 | unknown |
| 7 | TXv5v6-0169527 | v5v6 | Streptomyces |
| | | | ossamyceticus |
| 8 | S3167 (NRRL No. B-67735) | v1v9 | Variovorax |
| | | | paradoxus |
| 9 | S3167 (NRRL No. B-67735) | v1v8 | Variovorax |
| | | | paradoxus |
| 10 | S2492 (NRRL No. B-67736) | v1v9 | Variovorax |
| | | | paradoxus |
| 11 | S2492 (NRRL No. B-67736) | v1v8 | Variovorax |
| | | | paradoxus |
| 12 | S2876 (NRRL No. B-67448) | v1v9 | Niastella |
| | | | gongjuensis |
| 13 | S2876 (NRRL No. B-67448) | v1v9 | Niastella |
| | | | gongjuensis |
| 14 | S2876 (NRRL No. B-67448) | v1v9 | Niastella |
| | | | gongjuensis |
| 15 | S2876 (NRRL No. B-67448) | v1v8 | Niastella |
| | | | gongjuensis |
| 16 | S2876 (NRRL No. B-67448) | v1v8 | Niastella |
| | | | gongjuensis |
| 17 | S2876 (NRRL No. B-67448) | v1v8 | Niastella |
| | | | gongjuensis |
| 18 | S2695 (NRRL No. B-67444) | v1v9 | Arthrobacter |
| | | | globiformis |
| 19 | S2695 (NRRL No. B-67444) | v1v8 | Arthrobacter |
| | | | globiformis |
| 20 | S2695 (NRRL No. B-67444) | v1v9 | Arthrobacter |
| | | | globiformis |

### Example 3: Field Validation of Microbial Yield Enhancement

Field experiments were performed in 2018 across the United States, combining microbial candidates selected for association with increased plant performance for increased yield and yield stability under normal and moderate drought stress conditions.

The microbial treatments included consortia Bio17 (53167 Variovorax paradoxus, NRRL No. B-67735; S2492 Variovorax paradoxus, NRRL No. B-67736) and Bio18 (S2876Niastella gongjuensis, NRRL No. B-67448; S3167 Variovorax paradoxus, NRRL No. B-67735; S2492 Variovorax paradoxus, NRRL No. B-67736), applied as seed coatings using a carboxymethyl cellulose polymer on a set of four commercial maize hybrids.

Irrigation application was managed to impose drought stress during grain-filling at five sites, and the remaining sites received standard irrigation to avoid stress. In all locations, the crop was managed according to local commercial practices with effective control of weeds and pests. Yield data were collected in all locations. To evaluate the yield data, a mixed model framework was used to perform the single and multi-location analysis. In the single location analysis, main effects of hybrid and the microbial treatment are considered as fixed effects. The blocking factors such as replicates and incomplete block within replicates are considered as random. In the multi-location analysis, the main effect of microbial treatment and its interaction with location is considered a random effect. Yield analysis was conducted using ASREML (VSN International Ltd), Best Linear Unbiased Prediction (BLUP) (Cullis, B. R. et al. Biometrics 54, 1-18, 1998; Gilmour, A. R. et al. ASReml User Guide 3.0 2009; Gilmour, A. R., et al Biometrics 51, 1440-50, 1995).

Results from this experiment showed a positive impact on yield of both treatments across multiple hybrids and locations (See Table 2 hereinbelow). Bio17, a synthetic consortium of S3167 Variovorax paradoxus, NRRL No. B-67735; and S2492 Variovorax paradoxus. NRRL No. B-67735, had a positive yield effect in 70% of experiments, of which 11% were statistically significant (p<0.1), and the maximum increase observed was 10 bushel/acre yield advantage. Bio18, a synthetic consortium of S2876 Niastella gongjuensis, NRRL No. B-67448; S3167 Variovorax paradoxus, NRRL No. B-67735; and S2492 Variovorax paradoxus, NRRL No. B-67736, gave a positive yield effect in 64% of experiments, of which 11% were statistically significant (p<0.1) and the maximum increase seen was a 13 bushel/acre yield advantage.

TABLE 2
Effect of PGPM consortia on plant yield

| | | Yield Change (Difference between treated and untreated plants, average BLUP, | | | | | | | | | | |
|---|---|---|---|---|---|---|---|---|---|---|---|---|
| | | BU/Acre) in each location | | | | | | | | | | |
| | Consortium | Location Identifier | | | | | | | | | | |
| | Applied | (1) | (2) | (3) | (4) | (5) | (6) | (7) | (8) | (9) | (10) | (11) |
| HYBRID | Biol7* | 7.62 | 2.79 | 10.3 | 2.49 | −3.39 | 1.23 | −2.97 | 1.44 | 1.73 | −0.91 | −4.03 |
| 69 | Biol8** | 5.47 | 3.02 | 6.43 | 2.83 | | | 0.72 | 4.26 | 3.84 | 1.66 | 1.32 |
| HYBRID | Biol7 | −0.24 | −6.65 | 8.89 | 1.6 | 4.7 | 7.04 | 8.06 | 0.92 | 0.83 | −1.24 | −4.09 |
| 51 | Biol8 | −4.16 | −0.8 | −0.33 | −1.45 | | | −0.63 | −4.71 | −1.97 | 0.51 | 0.89 |
| HYBRID | Biol7 | 4.34 | 2.53 | −1.19 | 3.9 | −1.93 | 2.57 | 3.21 | 9.61 | 8.01 | 2.23 | 1.94 |
| 97 | Biol8 | 1.03 | 1.28 | −12.35 | −6.06 | | | −6.9 | 8.49 | 13.01 | 0.56 | 9.4 |
| HYBRID | Biol7 | 1.88 | 2.43 | −17.09 | 2.95 | −1.6 | 5.23 | 6.72 | 3.11 | 1.84 | −0.24 | 2.54 |
| 98 | Biol8 | −5.23 | 2.05 | 9.92 | 4.15 | | | 3.34 | 9.09 | −8.09 | −1.81 | 1.54 |

Table 2:
*Consortium Biol7: Strain S3167 + Strain S2492.
**Consortium Biol7: Strain S2876 + Strain S3167 + Strain S2492.
Location identifier: (1) CIYNBND2 (2) GCRLG2KN (3) JHRFAN23 (4) MRRFCNN2 (5) UCSFLAN2 (6) UCSNSTB2 (7) WNRN20A2 (8) WORFW82E (9) WORLB829 (10) WORLG82X (11) YKSLD201.
BLUP: Best Linear Unbiased Prediction.
BU: Bushel

### Example 4: Microbial Detection of Establishment

A total of 30 corn plants were collected at two time points corresponding to the V2-V3 and V4-V5 stage from a field in Woodland, Calif., and shaken to dislodge loose soil. Our sampling technique targeted the entire root system plus the tightly bound soil, referred to as the “root ball” (McPherson M R et al., J. Vis. Exp. 137:e57932. 2018. 10.3791/57932). To test the influence of increased organelle contamination (chloroplast and mitochondria) on our microbe detection sensitivity, the whole plant was also tested, including the root ball and the shoot. These “root ball” and “root-and-shoot” samples were stored at −80° C. prior to processing, ground to a fine powder in a freezer mill, and stored at −80° C.

To determine the limit of detection of target strains, a synthetic consortium of cultured microbes was added directly to the freezer milled root ball or root-and-shoot powder. The following strains were used: Arthrobacter globiformis strain S2695, NRRL No. B-67444: Niastella gongjuensis strain S2876, NRRL No. B-67448; Variovorax paradoxus strain S2492, NRRL No. B-67736; and Variovorax paradoxus strain S3167, NRRL No. B-67735. Cell counts were performed on all strains to determine the concentration of cells in each dilution. The strains were then diluted to equivalent concentrations, pooled to form a synthetic consortium, and then serially diluted to form seven dilution levels from 10⁸ cells/ml to 10² cells/ml. Each consortium dilution level was added in 50 μl aliquots to eight 200 mg replicates of homogenized root powder and eight 200 mg replicates of root-and-shoot powder. Accuracy of dilutions of the inoculant consortia was validated by quantitative polymerase chain reaction (qPCR).

For determination of the background community, or negative controls, 200 mg of the homogenized root and root-and-shoot powder was used without any addition of the synthetic consortium. For positive controls, we used three dilutions of the consortium at 10⁶ cells/ml, 10⁴ cells/ml, and 10² cells/ml without addition of the freezer milled powder. All controls were prepared in replicates of eight.

DNA was extracted using ZymoBiomics® DNA Extraction Kits (Cat. D4300). To determine the most accurate and sensitive establishment detection methodology, 16S microbial community profiling was done using two library preparation and processing methods. The first method used Illumina® HiSeq 16s rDNA amplicon sequencing, utilizing a two-step PCR and dual indexing strategy. DNA was amplified using the forward primer TX9 (5′-GGATTAGAWACCCBGGTAGTC-3′ (SEQ ID NO: 21), Ashby et al. 2007 AME 73(14):4532-4542) and reverse primer CMC-03R (5′-TCACRRYACGARCTGRCG-3′ (SEQ ID NO: 22)) to select for the V5V6 region of 16S rRNA. The second method used Loop Genomics® library preparation for long-read, full length 16S rRNA sequencing (LoopSeq™ 16S Microbiome 24-plex Kit). Two libraries of 24 samples each were multiplexed per lane and sequenced on an Illumina® HiSeq 4000 sequencer; reads were processed by the Loop Genomics® pipeline.

Sequences from all pipelines were processed as described in Patin et al. (Microb. Ecol. 65:709-719, 2013, and U.S. Pat. No. 9,593,382). The Loop Genomics® contigs were trimmed to V1V8 and V5V6 regions and Illumina®-sequenced amplicons were trimmed to V5V6 only. The trimming rules are shown in Table 3. Sequences comprising low quality bases (below Q20) were removed. The trimmed sequences were referred to as “tags.” Tags with identical sequences were counted, and relative abundances of each tag calculated.

TABLE 3
Pattern recognition rules for
sequence trimming to regions.

| | V1V8 | V5V6 |
|---|---|---|
| Start of | AGAGTTTGAT (SEQ | GGATTAGA (SEQ ID |
| primer | ID NO: 23) | NO: 24) |
| End of primer | TGGCTCAG (SEQ ID | GGTAGTC (SEQ ID |
| | NO: 27) | NO: 28) |
| Primer size | 13 | 21 |
| Trim at | TGNACNCACNGCCCG | ATGGCTGTCGTCAG |
| | TC (SEQ ID NO: 25) | CT (SEQ ID NO: 26) |
| Number of | 3 | 5 |
| mismatches | | |
| for rejection | | |
| Shortest | 1000 | 230 |
| sequence size | | |
| Longest | 2200 | 290 |
| sequence size | | |

Tags were assigned taxonomy using RDP Classifier v11. All tags with a genus classification of Streptophyta or Chloroplast were counted as chloroplast. All sequences with a phylum classification of Proteobacteria and undetermined class, order, family, genus, and species were counted as mitochondria. Tags counted as chloroplast and mitochondria, or organelles, were excluded from further analysis.

To test the method for identification of microbial establishment, homogenized environmental samples were inoculated with serial dilutions of a microbial consortium. DNA was then extracted and microbial community 16S rDNA profiling was performed using Loop Genomics® and Illumina® HiSeq sequencing technologies.

Illumina® HiSeq sequencing resulted in an average 269,213 (stdev 106,524) V5V6 tags per sample that passed the quality controls. Loop Genomics® produced an average of 15,060 (stdev 9,880) synthetic reads per sample. Of those, on average 7,850 (stdev 6,280) synthetic reads per sample passed our filtering criteria for V1V8 region.

Prior to calculation of microbial abundances, organelle sequences were removed from all samples. Far fewer organelles were removed from the samples with root ball material (23.4% to 38%) than the samples with root-and-shoot material (37.9% to 77.9%).

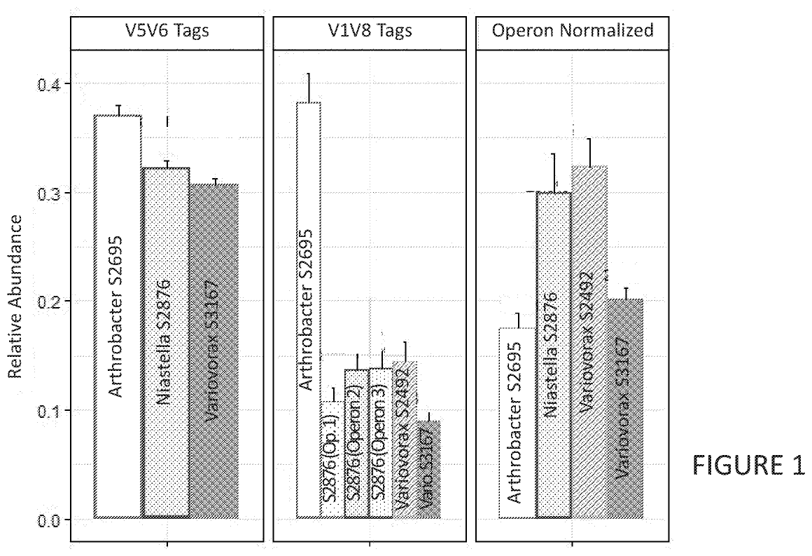

The four inoculant synthetic consortium strains corresponded to three V5V6 tags. Two Variovorax strains were represented by a single tag (i.e. had identical sequences in the V5V6 region of the 16S rDNA). Other strains were each represented by a single tag. Conversely, sequencing the larger V1V8 region allowed for increased specificity, and the synthetic community corresponded to six V1V8 tags. For Niastella, V1V8 sequences were not only unique between different strains, but different operons within the same strain. Other strains were represented by exactly a single tag each (See FIG. 1).

Negative control samples, consisting of only plant powder with no synthetic consortium added, show the relative abundances of the consortium microbes as they naturally exist in the background community. The background relative abundances of V5V6 tags corresponding to added microbes of the synthetic community are shown in Table 4. The background levels for all tags were non-zero, lowest for Niastella, intermediate for Variovorax and highest for Arthrobacter. Relative abundances were between 1.2% and 2.15% in the root ball.

The background relative abundances of V1V8 tags corresponding to added microbes of the synthetic community are shown in Table 5. The background was 0 for Niastella and was below 2 per 10,000 tags for both Variovorax tags. Arthrobacter had the highest background levels, reaching 0.45% in the root.

TABLE 4
Background relative abundances (1 per 100) of
synthetic community V5V6 tags in native samples

| | |
|---|---|
| S16 rRNA | Root |

| | Region/Tag | Average | STDEV |
|---|---|---|---|
| Niastella | TXV5V6-2136654 | 1.20 | 2.56 |
| Variovorax | TXV5V6-0053056 | 2.12 | 4.53 |
| Arthrobacter | TXV5V6-0586156 | 2.15 | 2.72 |

TABLE 5
Background relative abundances (1 per 100) of synthetic
community V1V8 tags in the native samples

| | |
|---|---|
| S16 rRNA | Root |

| | region/Tag | Average | STDEV |
|---|---|---|---|
| Variovorax A | TXV1V8-000044452 | 0.0049 | 0.0138 |
| Variovorax B | TXV1V8-000001064 | 0.0189 | 0.0152 |
| Niastella 1 | TXV1V8-000018596 | 0 | n/a |
| Niastella 2 | TXV1V8-000018597 | 0 | n/a |
| Niastella 3 | TXV1V8-000018595 | 0 | n/a |
| Arthrobacter | TXV1V8-000000739 | 0.451 | 0.1007 |

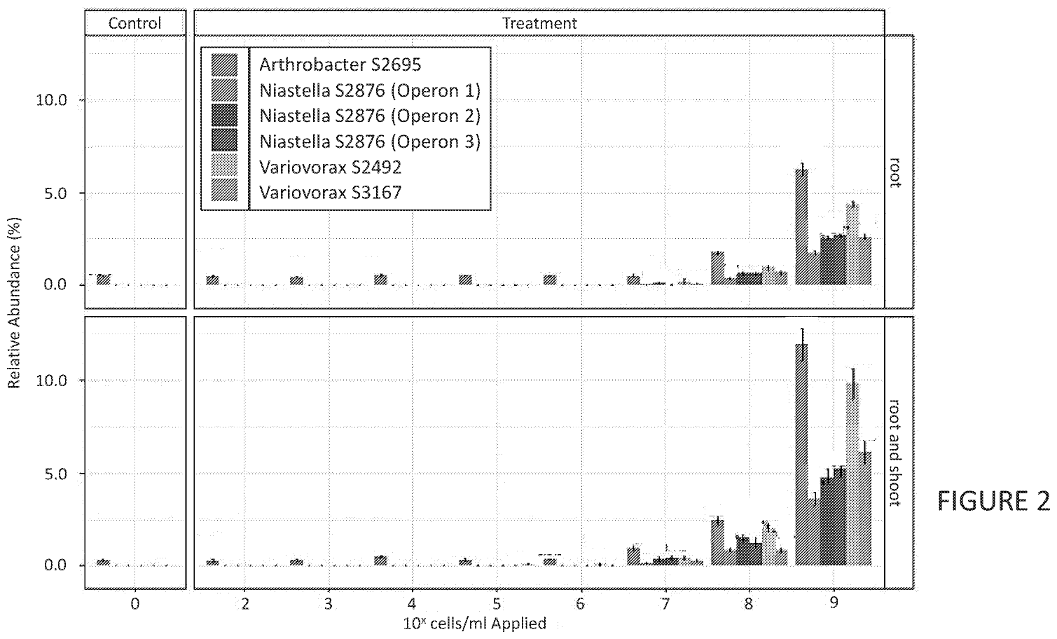

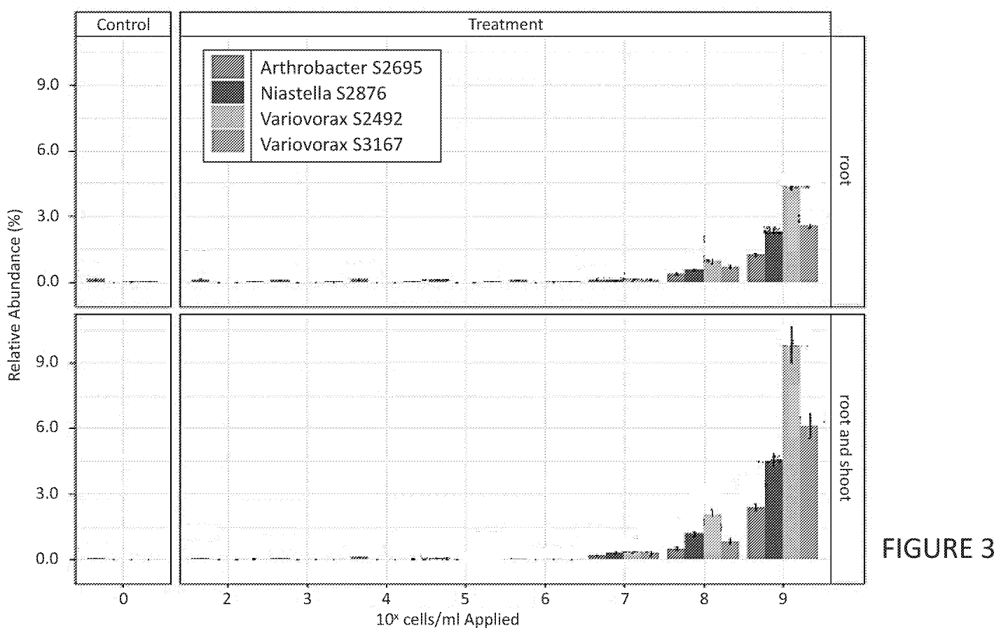

The background abundances dictate the level of added microbial presence that must be achieved before elevated counts could be observed. FIGS. 2 and 3 show the observed abundances of the synthetic community across the different concentrations in which it was added to freeze-milled plant powder. Both V5V6 and V1V8 technologies allowed identification of elevated microbial abundances relative to the background. Using the V5V6 method allowed two concentrations (10⁷ and 10⁶ cells/ml) to be clearly seen above background. The dilutions became indistinguishable from the background for Niastella gongjuensis (S2876 B-67448) at 10⁵ cell/ml and at 10⁷ cells/ml for Arthrobacter globiformis (S2695, B-67444). The background presence of Variovorax paradoxus (S2492, B-67736 and S3167, B-67735) was intermediate, allowing identification at 10⁶ cells/ml. Using the V1V8 method allowed another level of dilution (10⁵ cells/ml) for the two Variovorax strains (S2492, B-67736 and S3167, B-67735) to be distinguished between them and from the background.

### Example 5: Clustering of Microbial Strains Using Strain-Specific Genomic-Markers

### Experimental Procedures

DNA fragments with lengths ranging from 200 bp to 500 bp from genomes of the microbial strains of certain embodiments of this invention, were screened against the NCBI nucleotide database using NCBI local alignment tool BLASTN (NCBI-blast-2.7.1+). Criteria for declaring a microbial strain-specific marker are at least 90% coverage with at least 95% local sequence identity. 1-5 microbial strain-specific markers were selected for certain microbial strains described in this invention as presented in Table 6 hereinbelow.

TABLE 6
Marker SEQ ID NOs per strain

| | | | Number of | |
|---|---|---|---|---|
| | | | Microbial | Marker Sequences |
| Microbial | | | strain- | length, concordant |
| strain | | Marker SEQ | specific | with Marker |
| number | Organism | ID NOs | markers | SEQ ID NOs |
| S3167 | Variovorax | 29; 30; 31; | 5 | 228; 296; 263; |
| | paradoxus | 32; 33 | | 244; 242 |
| S2492 | Variovorax | 34; 35; 36; | 5 | 490; 472; 352; |
| | paradoxus | 37; 38 | | 283; 276 |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A microbial preparation comprising at least one isolated microbial strain, a functional homolog thereof or an enriched culture of same, wherein the isolated microbial strain is selected from the group consisting of:
a. strain S3167, the strain being selected from the group consisting of:
i. a strain deposited under Accession Number NRRL No. B-67735;
ii. a strain comprising at least one 16S-rRNA sequence comprising a nucleic acid sequence selected from the group consisting of SEQ ID NO:1, SEQ ID NO:8, and SEQ ID NO:9; and
iii. a strain comprising at least one genomic marker comprising the nucleic acid sequence set forth in any one of SEQ ID NO:29, SEQ ID NO:30, SEQ ID NO:31, SEQ ID NO:32, and SEQ ID NO:33;
b. strain S2492, the strain being selected from the group consisting of:
i. a strain deposited under Accession Number NRRL No. B-67736;
ii. a strain comprising at least one 16S-rRNA sequence comprising a nucleic acid selected from the group consisting of SEQ ID NO:1, SEQ ID NO:10, and SEQ ID NO:11; and
iii. a strain comprising at least one genomic marker comprising the nucleic acid sequence set forth in any one of SEQ ID NO:34, SEQ ID NO:35, SEQ ID NO:36, SEQ ID NO:37, and SEQ ID NO:38;
c. strain S2441, the strain comprising a 16S-rRNA sequence comprising the nucleic acid sequence set forth in SEQ ID NO:2;
d. strain S2876, the strain comprising a 16S-rRNA sequence comprising the nucleic acid sequence set forth in SEQ ID NO:3;
e. strain S2550, the strain comprising a 16S-rRNA sequence comprising the nucleic acid sequence set forth in SEQ ID NO:4; and
f. a strain comprising a 16S-rRNA sequence comprising a nucleic acid sequence selected from the group consisting of SEQ ID NO:5, SEQ ID NO:6, or SEQ ID NO:7.

2-5. (canceled)

6. The microbial preparation according to claim 1, wherein at least one of the following exists:
a. the functional homolog of microbial strain S3167 comprises at least one of: a 16S-rRNA sequence at least 85% identical to SEQ ID NO:1; a 16S-rRNA sequence at least 97.5% identical to SEQ ID NO:8; a 16S-rRNA sequence at least 94.5% identical to SEQ ID NO:9; and a genomic nucleic acid marker having at least 95% local identity to a nucleic acid sequence set forth in any one of SEQ ID NO:29, SEQ ID NO:30, SEQ ID NO:31, SEQ ID NO:32, and SEQ ID NO:33 over 90% coverage;
b. the functional homolog of microbial strain S2492 comprises at least one of: a 16S-rRNA sequence at least 85% identical to SEQ ID NO:1; a 16S-rRNA sequence at least 97.5% identical to SEQ ID NO:10; a 16S-rRNA sequence at least 94.5% identical to SEQ ID NO:11; and a genomic nucleic acid marker having at least 95% local identity to a nucleic acid sequence set forth in any one of SEQ ID NO:34, SEQ ID NO:35, SEQ ID NO:36, SEQ ID NO:37, and SEQ ID NO:38 over 90% coverage; and
c. the functional homolog is selected from the group consisting of: a functional homolog of microbial strain S2441 comprising a 16S-rRNA sequence at least 85% identical to SEQ ID NO:2; a functional homolog of microbial strain S2876 comprising a 16S-rRNA sequence at least 85% identical to SEQ ID NO:3; a functional homolog of strain S2550 comprising a 16S-rRNA sequence at least 85% identical to SEQ ID NO:4; and a functional homolog comprising a 16S-rRNA sequence at least 85% identical to SEQ ID NO:5, SEQ ID NO:6, or SEQ ID NO:7.

7-12. (canceled)

13. The microbial preparation according to claim 1, wherein said microbial preparation comprises a combination of at least two of the isolated microbial strains, functional homologs thereof or the enriched cultures of same.

14. The microbial preparation according to claim 13, wherein said microbial preparation comprises the isolated Variovorax paradoxus strains S3167 and S2492.

15. The microbial preparation according to claim 14, wherein said microbial preparation further comprises the isolated Niastella gongjuensis strain S2876.

16. A composition comprising the microbial preparation according to claim 1.

17-18. (canceled)

19. The composition according to claim 16, wherein said composition further comprises at least one additional microbial strain selected from the group consisting of P0035_B2 (also referred to as S2145; NRRL Deposit No. B-67091), P0020_B1, P0047_A1 (also referred to as S2284; NRRL Deposit No. B-67102), P0033_E1 (also referred to as S2177), P0032_A8 (also referred to as S2181; NRRL Deposit No. B-67099), P0049_E7, P0042_A8 (also referred to as S2167), P0042_D5 (also referred to as S2165), P0042_B2 (also referred to as S2168; NRRL Deposit No. B-67096), P0042_B12 (also referred to as S2189), P0042_C2 (also referred to as S2173; NRRL Deposit No. B-67098), P0042_D10 (also referred to as S2172; NRRL Deposit No. B-67097), P0044_A3 (also referred to as S2476), P0018_A11, P0044_A5, P0047_E2, P0047_C1, P0038_D2 (also referred to as S2166), P0042_E1, P0047_E8, P0018_A1, P0058_B9 (also referred to as S2159; NRRL Deposit No. B-67092), P0054_E8 (also referred to as S2161; NRRL Deposit No. B-67094), P0054_F4 (also referred to as S2164), P0057_A3 (also referred to as S2160; NRRL Deposit No. B-67093), P0061_E11 (also referred to as S2142), P0019_A12 (also referred to as S2163; NRRL Deposit No. B-67095), P0147_D10 (also referred to as S2291; NRRL Deposit No. B-67104), P0147_G10 (also referred to as S2292; NRRL Deposit No. B-67105), P0160_F7 (also referred to as S2351), P0140_C10 (also referred to as S2300; NRRL Deposit No. B-67107), S2387, P0157_G5 (also referred to as S2303; NRRL Deposit No. B-67108, P0160_E1 (also referred to as S2374), P0134_G7 (also referred to as S2280), S2384 (NRRL Deposit No. B-67112), S2275 (NRRL Deposit No. B-67101), S2278, S2373 (NRRL Deposit No. B-67109), S2370, S2293 (NRRL Deposit No. B-67106), S2382 (NRRL Deposit No. B-67111), P0132_A12, P0132_C12, P0140_D9, P0173_H3 (also referred to as S2404), S2385 (NRRL Deposit No. B-67113), S2197 (NRRL Deposit No. B-67100), S2285 (NRRL Deposit No. B-67103), S2477, S2376, S2420, S2424, S2445, S2333, S2329, S2327, S2330, S2423 (NRRL Deposit No. B-67115), S2435, S2158, S2437, S2332, S2521, S2228, S2473, P0156_G2, P0154_G3, S2487, S2488, S2421 (NRRL Deposit No. B-67114), P0105_C5, P0154_H3, P0156_G1, S1112 (NRRL Deposit No. B-67090), S2375 (NRRL Deposit No. B-67110), S2669 (NRRL Deposit No. B-67117), S2651, S2652, S2653, S2654, S2655, S2656, S2668, S2644 (NRRL Deposit No. B-67116), S2328, S2646, S2834 (NRRL Deposit No. B-67441), S2381 (NRRL Deposit No. B-67442), S2543 (NRRL Deposit No. B-67443), S2695 (NRRL Deposit No. B-67444), S2700 (NRRL Deposit No. B-67445), S2145-2 (NRRL Deposit No. B-67331), S2292-2 (NRRL Deposit No. B-67332), S2300-2 (NRRL Deposit No. B-67333), S2303-2 (NRRL Deposit No. B-67334), S2375-2 (NRRL Deposit No. B-67335), S2382-2 (NRRL Deposit No. B-67336), S2423-2 (NRRL Deposit No. B-67337), S2669-2 (NRRL Deposit No. B-67338), a functional homolog thereof or a strain derived therefrom.

20. The composition of claim 16, wherein said composition further comprises a plant or a plant seed.

21. The composition of claim 20, wherein the plant or the plant seed comprises at least one genetically modified cell conferring enhancement of at least one trait compared to a non-modified plant or plant seed.

22. (canceled)

23. The composition of claim 16, wherein said composition is an agricultural composition further comprising agriculturally acceptable diluents or carriers.

24-26. (canceled)

27. The composition of claim 23, wherein the carrier is a plant seed and wherein said composition is a seed coating formulation.

28. A plant or a plant seed comprising the microbial preparation according to claim 1 or a composition comprising same.

29. (canceled)

30. The plant or plant seed according to claim 28, wherein said plant or plant seed comprises at least one genetically modified cell conferring enhancement of at least one trait compared to a non-modified plant or plant seed.

31. A method for enhancing at least one of the health, growth and yield of a plant, comprising applying to the plant, part thereof, or the plant growth medium an effective amount of the microbial preparation according to claim 1 or a composition comprising same.

32. A method for preventing, inhibiting or ameliorating the development of a plant disease caused by a plant pathogen, comprising applying to the plant, part thereof, or the plant growth medium an effective amount of the microbial preparation according to claim 1 or a composition comprising same.

33. (canceled)

34. The method of claim 32, wherein the effective amount of the microbial preparation comprises at least 1×10² CFU of the at least one isolated microbial strain or functional homolog thereof.

35-39. (canceled)

40. A method for treating a plant seed, comprising exposing or contacting the plant seed with an effective amount of the microbial preparation according to claim 1 or a composition comprising same.

41. The method of claim 40, wherein exposing or contacting the plant seed with the effective amount of the microbial preparation or composition comprising same is concomitant to said seed priming.

42. The method of claim 40, wherein the effective amount of the microbial preparation comprises at least 1×10² CFU of the at least one isolated microbial strain.

43. The method of claim 31, wherein the effective amount of the microbial preparation comprises at least 1×10² CFU of the at least one isolated microbial strain or functional homolog thereof.
